(19) United States
(12) Patent Application Publication
(10) Pub. No.: US 2014/0316217 A1
(43) Pub. Date: Oct. 23, 2014

(54) SYSTEM AND METHOD FOR MONITORING ANESTHESIA AND SEDATION USING MEASURES OF BRAIN COHERENCE AND SYNCHRONY

(71) Applicants: Patrick L. Purdon, Somerville, MA (US); Laura D. Lewis, Cambridge, MA (US); Oluwaseun Akeju, Dorchester, MA (US); Emery N. Brown, Brookline, MA (US)

(72) Inventors: Patrick L. Purdon, Somerville, MA (US); Laura D. Lewis, Cambridge, MA (US); Oluwaseun Akeju, Dorchester, MA (US); Emery N. Brown, Brookline, MA (US)

(21) Appl. No.: 14/260,006

(22) Filed: Apr. 23, 2014

Related U.S. Application Data
(60) Provisional application No. 61/815,141, filed on Apr. 23, 2013.

(51) Int. Cl. A61B 5/00; A61B 5/145; A61B 5/0476

(52) U.S. Cl. CPC A61B 5/4821; A61B 5/7275; A61B 5/0476; A61B 5/14542
USPC 600/301; 600/300

(57) ABSTRACT

A system and method for monitoring and controlling the administration of at least one drug having anesthetic properties are provided. In certain embodiments, the method includes assembling physiological data, obtained from a plurality of sensors placed on a subject, into sets of time-series data, separating, from the sets of time-series data, a plurality of low frequency signals, and determining, from the plurality of low frequency signals, at least one of coherence information and synchrony information. The method can also include identifying, using the at least one of the coherence information and the synchrony information, spatiotemporal signatures indicative of at least one of a current state and a predicted future state of the patient consistent with the administration of at least one drug having anesthetic properties and generating a report indicating at least one of the current state and the predicted future state of the patient induced by the drug.

### CROSS-REFERENCE TO RELATED APPLICATIONS

This application is based on, claims priority to, and incorporates herein by reference in its entirety, U.S. Provisional Application Ser. No. 61/815,141, filed Apr. 23, 2013, and entitled “SYSTEM AND METHOD FOR MONITORING GENERAL ANESTHESIA AND SEDATION USING ELECTROENCEPHALOGRAM MEASURES OF BRAIN COHERENCE AND SYNCHRONY.”

### STATEMENT REGARDING FEDERALLY SPONSORED RESEARCH

This invention was made with government support under DP2-OD006454, TR01-GM104948, and T32GM007592 awarded by the National Institutes of Health. The government has certain rights in the invention.

### BACKGROUND OF THE INVENTION

The present invention generally relates to systems and methods for monitoring and controlling a state of a patient and, more particularly, to systems and methods for monitoring and controlling a state of a patient receiving a dose of anesthetic compound(s) or, more colloquially, receiving a dose of “anesthesia.”

The practice of anesthesiology involves the direct pharmacological manipulation of the central nervous system to achieve the required combination of unconsciousness, amnesia, analgesia, and immobility with maintenance of physiological stability that define general anesthesia. With increasing clinical use of anesthetics and number of compounds with anesthetic properties growing, scientific understanding of the operation of the body when under anesthesia is increasingly important. For example, a complete understanding of the effects of anesthesia on patients and operation of the patient's brain over the continuum of “levels” of anesthesia is still lacking. Tools used by clinicians when monitoring patients receiving a dose of anesthesia include electroencephalogram-based (EEG) monitors, developed to help track the level of consciousness of patients receiving general anesthesia or sedation in the operating room and intensive care unit.

However, there continues to be a clear need for systems and methods to accurately monitor and quantify patient states and based thereon, provide systems and methods for controlling patient states during administration of anesthetic compounds.

### SUMMARY OF THE INVENTION

The present invention overcomes drawbacks of previous technologies by providing systems and methods for monitoring and controlling brain states related to the administration and control of anesthetic compounds, using measures of brain coherence and synchrony.

In one aspect of the invention, a system for monitoring a patient experiencing an administration of at least one drug having anesthetic properties is provided. The system includes a plurality of sensors configured to acquire physiological data from the patient and a user interface configured to receive an indication of at least one of a characteristic of the patient and the at least one drug having anesthetic properties The system also includes at least one processor configured to receive the physiological data from the plurality of sensors and the indication from the user interface, assemble the physiological data into sets of time-series data, and separate, from the sets of time-series data, a plurality of low frequency signals. The at least one processor is also configured to determine, from the plurality of low frequency signals, at least one of coherence information and synchrony information and identify, using the at least one of the coherence information and the synchrony information, spatiotemporal signatures indicative of at least one of a current state and a predicted future state of the patient consistent with the administration of at least one drug having anesthetic properties. The at least one processor is further configured to generate a report indicating at least one of the current state and the predicted future state of the patient induced by the drug.

In another aspect of the disclosure, a method for monitoring a patient experiencing an administration of at least one drug having anesthetic properties is provided. The method includes arranging a plurality of sensors configured to acquire physiological data from a patient, reviewing the physiological data from the plurality of sensors and the indication from the user interface and assembling the physiological data into sets of time-series data. The method also includes separating, from the sets of time-series data, a plurality of low frequency signals, determining, from the plurality of low frequency signals, at least one of coherence information and synchrony information and identifying, using the at least one of the coherence information and the synchrony information, spatiotemporal signatures indicative of at least one of a current state and a predicted future state of the patient consistent with the administration of at least one drug having anesthetic properties. The method further includes generating a report indicating at least one of the current state and the predicted future state of the patient induced by the drug.

The foregoing and other advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will hereafter be described with reference to the accompanying drawings, wherein like reference numerals denote like elements.

FIG. 1A is a schematic block diagram of a traditional anesthetic compound monitoring and control system that depends completely upon a clinician.

FIG. 1B is a schematic illustration of a traditional closed-loop anesthesia delivery (CLAD) system.

FIGS. 2A and 2B are block diagrams of example monitoring and control systems in accordance with the present disclosure.

FIG. 3A is an illustration of an example monitoring and control system in accordance with the present disclosure.

FIG. 3B is an illustration of an example portable monitoring system in accordance with the present disclosure.

FIG. 3C is an illustration of an example display for the monitoring and control system of FIG. 3A

FIG. 4 is a flow chart setting forth the steps of a monitoring and control process in accordance with the present disclosure.

FIG. 5A is a flow chart setting forth steps of a process for determining a brain state of a patient, in accordance with the present disclosure.

FIG. 5B is an example system for use in determining a brain state of a patient, in accordance with the present disclosure.

FIG. 6 is a flow chart setting forth steps of a method for monitoring a patient in accordance with the present disclosure.

FIG. 7 is a graph illustrating example effects on spike rates for different patients during propofol-induced loss of consciousness (LOC).

FIG. 8A is a graphical example illustrating slow oscillation power increase at LOC for different patients.

FIG. 8B is a spectrogram example illustrating power changes at LOC over a frequency range.

FIG. 9 is a graphical example illustrating that spikes become phase-coupled to the slow oscillation at LOC.

FIGS. 10A through 10C are charts illustrating examples that slow oscillations in distant electrocorticogram (“ECoG”) channels have variable phase offsets.

FIGS. 11A through 11E provide a graphical example illustrating that slow oscillations are asynchronous across a cortex and are associated with ON/OFF states.

FIGS. 12A through 12C provide graphs illustrating that spikes occur in brief ON periods that maintain inter-unit structure.

FIGS. 13A through 13E provide graphs illustrating that spike activity is associated with modulations in slow oscillation, morphology, and gamma power.

FIG. 14A is a graphical example illustrating representative spectrograms and the time-domain electroencephalogram signals during dexmedetomidine sedation, propofol-induced sedation and general unconsciousness.

FIG. 14B is a graphical example illustrating representative ten-second electroencephalogram traces of dexmedetomidine sedation, propofol-induced sedation and general unconsciousness, showing shared EEG dynamics within the slow and alpha/spindle frequencies.

FIGS. 15A through 15C are graphical illustrations of example frontal midline group level spectrogram data and spectral power differences between dexmedetomidine baseline and sedation.

FIGS. 16A through 16C are graphical illustrations of example frontal midline group level spectrogram data and spectral power differences between propofol baseline, sedation and unconsciousness.

FIG. 17 shows a graphical illustrations of example frontal midline group level spectral power differences between propofol and dexmedetomidine sedation.

FIG. 18A is a visual representation of an example frontal electrode placement for use in a coherence analysis, in accordance with the present disclosure.

FIGS. 18B and 18C are graphical illustrations of example signal data illustrating how coherograms quantify relationships between signals, in distinction of spectrograms.

FIGS. 19A through 19C are graphical illustrations of example group level coherogram data and coherence differences between dexmedetomidine baseline and sedation.

FIGS. 20A through 20E are graphical illustrations of example group level coherogram data and coherence differences between propofol baseline, sedation and unconsciousness.

FIGS. 21A and 21B are graphical illustrations of example group level coherence differences between propofol and dexmedetomidine sedation.

FIGS. 22A and 22B is a graphical example illustrating representative ten-second electroencephalogram traces of sevoflurane and propofol general anesthesia.

FIGS. 23A through 23C are graphical illustrations of example group level spectrogram data and spectral power differences between sevoflurane and propofol general anesthesia.

FIGS. 24A through 24C are graphical illustrations of example group level coherogram data and coherence differences between sevoflurane and profofol general anesthesia.

### DETAILED DESCRIPTION

Despite major advances in identifying common molecular and pharmacological principles that underlie anesthetic drugs, it is not yet clear how actions at different molecular targets affect large-scale neural dynamics to produce unconsciousness. As such, anesthesiologists are typically trained to recognize the effects of anesthesia and extrapolate an estimate of the “level” of anesthetic influence on a given patient based on the identified effects of the administered anesthesia.

Using proprietary algorithms that combine spectral and entropy measurements, monitoring systems typically provide feedback through partial or amalgamized representations of the acquired signals. For example, many systems quantify the physiological responses of the patient receiving the dose of anesthesia and, thereby, convey the patient's depth of anesthesia, through a single dimensionless index. However, indices currently utilized generally relate indirectly to the level of consciousness, and given that different drugs act through different neural mechanisms, and produce different electroencephalogram (“EEG”) signatures, associated with different altered states of consciousness, such approaches may be qualitative at best. Consequently, some EEG-based depth of anesthesia indices have been shown to poorly represent a patient's brain state, and moreover show substantial variability in underlying brain state and level of awareness at similar numerical values within and between patients. Not surprising, compared to non depth-of-anesthesia monitor based approaches, these monitors have been ineffective in reducing the incidence of intra-operative awareness.

In practice, one common process that clinicians use is to monitor EEG display to identify indications of “burst suppression.” Burst suppression is an example of an EEG pattern that can be observed when the brain has severely reduced levels of neuronal activity, metabolic rate, and oxygen consumption. For example, burst suppression is commonly seen in profound states of general anesthesia. One example of a profound state of a patient under general anesthesia is medical coma. The burst suppression pattern often manifests as periods of bursts of electrical activity alternating with periods during which the EEG is isoelectric or suppressed. A variety of clinical scenarios require medical coma for purposes of brain protection, including treatment of uncontrolled seizures—status epilepticus—and brain protection following traumatic or hypoxic brain injury, anoxic brain injuries, hypothermia, and certain developmental disorders. Burst suppression represents a specific brain state resulting from such injuries, disorders, or medical interventions.

Traditional systems and methods that attempt to quantify burst suppression proceeds in two steps. First, characteristics of burst suppression are identified in the acquired data and the burst and suppression events are segregated or separated from EEG artifacts by conversion into a binary time-series format. Second, these systems and methods attempt to quantify the level of burst suppression. For example, some commercially available brain monitoring devices use a so-called “burst suppression ratio” (“BSR”) as part of an algorithm to identify and track the state of burst suppression, where the BSR is a quantify related to the proportion of time, in a given time interval, that the EEG signal is designated as suppressed.

Although the importance of quantitatively analyzing burst suppression using, for example, a metric like BSR is broadly appreciated, in some instances, analyzing burst suppression by itself may not accurately indicate a state of consciousness. For example, even though binary values can be computed on intervals as short as 100 millisececonds or even every millisecond, it is not unusual to use several seconds of these binary values to compute the BSR. This assumes that the brain state remains stable throughout the period during which the BSR is being computed. When the level of brain activity is changing rapidly, such as with induction of general anesthesia, hypothermia, or with rapidly evolving disease states, this assumption may not hold true. Instead, the computation of the level of burst suppression should match the resolution at which the binary events are recorded. Unfortunately, this reflects a practical quandary for the algorithm designer. Namely, the design cannot calculate a BSR without a determined time interval, but the true interval would be best selected with knowledge of the BSR to be calculated.

To further compound the difficulties of using such BSR algorithms clinically, different manufactures use different segmentation algorithms to convert the EEG into a binary time-series. Accordingly, different devices from different manufactures produce different BSR estimates. Comparing results across devices/manufacturer's is often challenging. As a further clinical challenge, for any of the situations in which burst suppression is tracked quantitatively, an important objective is to make formal statistical comparisons at different points in time. However, the statistical properties of the BSR estimated by averaging the binary events over several second intervals have not been described. As a consequence, there is no principled way to use the current BSR estimates in formal statistical analyses of burst suppression. That is, there is a lack of formal statistical analyses and prescribed protocols to implement formal statistical analyses to be able to state with a prescribed level of certainty that two or more brain states differ using current BSR protocols.

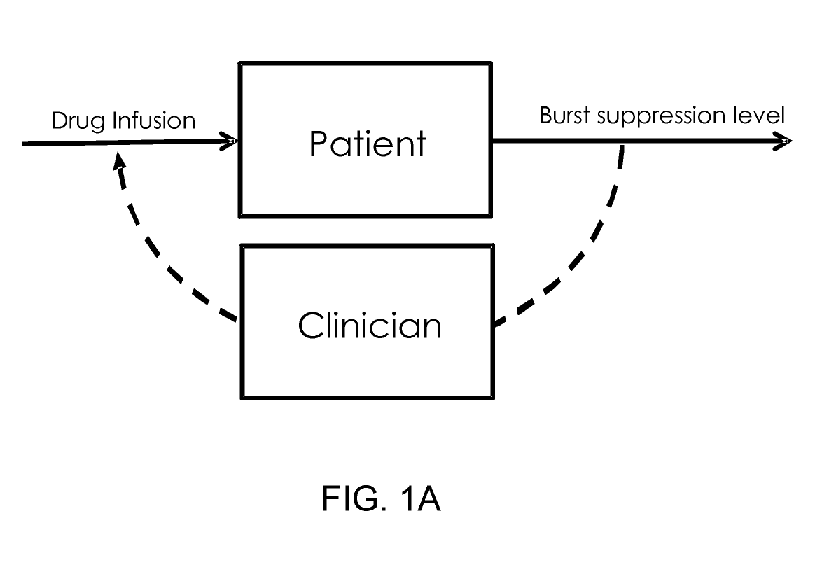

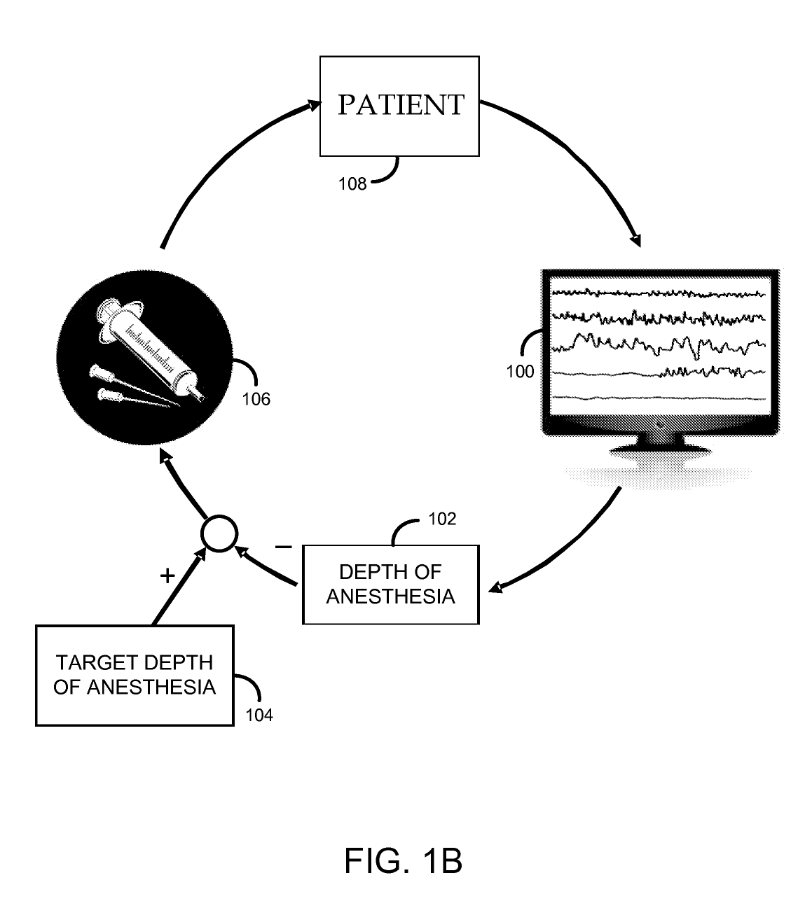

The shortcomings of these monitoring systems is compounded by the fact that they are often used as the information source on which clinicians make decisions. For example, referring to FIG. 1A, a simplified schematic is illustrated showing that a “drug infusion” including a dose of anesthesia is delivered to a patient. Feedback from the patient is gathered by a monitoring system such as described above that attempts to identify and quantify burst suppression by providing an indication of “burst suppression level”. The “burst suppression level” is generally the amount of burst suppression perceived by the clinician looking at the monitor display. This “burst suppression level” then serves as the input to a clinician that serves as the control of a feedback loop by adjusting the drug infusion levels based on the indicated “burst suppression level.” This simplified example illustrates that errors or general inaccuracies in the “burst suppression level” indicated by the monitoring system and/or erroneous interpretations or assumptions by the clinician can exacerbate an already inexact process of controlling the drug infusion process. Such imprecision may be tolerable in some situations, but is highly unfavorable in others.

For example, in some clinical settings, it may be desirable to place a patient in a so-called “medical coma.” To do so, burst suppression is induced by manually tuning drug infusion to meet certain specifications. Control of these infusions requires the nursing staff to monitor, frequently by eye, the infusion pump and the EEG waveform, and to titrate the infusion rate of the anesthetic drug to achieve and maintain the desired EEG pattern. It is impractical for the nursing staff to provide a continuous assessment of the EEG waveform in relation to the rate of drug infusion in such a way to maintain tight control of the patient's desired brain state.

With these clinical challenges recognized, some have attempted to develop feedback and control systems to aid the clinician. For example, Bickford proposed an EEG-based, closed loop anesthetic delivery (“CLAD”) system more than 60 years ago. For example, a simplified schematic diagram of an early CLAD system is provided in FIG. 1B. Bickford's original CLAD system of the 1950s used EEG content 100 in specific frequency bands as the control signal that indicated a current “depth of anesthesia” 102. The depth of anesthesia 102 was compared to a “target depth of anesthesia” 104, which determined with the drug infusion 106 should be increased or decreased. As such, a closed loop system was proposed to control the anesthetic delivered to the patient 108.

Later incarnations of the proposed CLAD systems used more sophisticated EEG analysis. For example, instead of simply relying on specific frequency bands as the control signal, systems were proposed that used metrics, such as the median frequency and the spectral edge, or the 50th and 95th quantiles of the power spectrogram, respectively. Studies observed a strong relationship between frequency content and its associated range and the corresponding depth of general anesthesia. Other possible control signals that were proposed included evoked potentials, or physiological responses, such as heart rate and blood pressure. Though commercial development of such systems did not begin in earnest until the 1980's, there have now been many clinical studies on the use of CLAD systems in anesthesiology practice and a system for sedation not using EEG is now commercially available.

Although CLAD systems have been around for many years and they are now used in anesthesiology practice outside of the United States, recent reports suggest that several problems with these systems have not been fully addressed. First, it has been recognized since 1937 that EEG patterns can serve as an indicator of brain state under general anesthesia. To date, sufficiently detailed quantitative analyses of the EEG waveform have not been performed to produce well-defined markers of how different anesthetic drugs or combinations of drugs alter the states of the patient and how such variations manifest in EEG waveforms and other physiological characteristics.

In an attempt to combat such problems, the so-called Bispectral Index (“BIS”) has been used an EEG-based marker to track brain state under general anesthesia and to provide a control signal for CLAD systems. BIS is derived by computing spectral and bispectral features of the EEG waveform. The features are input to a proprietary algorithm to derive an index between 0 and 100, in which 100 correspond to fully awake state with no drug effects and 0 corresponds to the most profound state of coma. As referenced above, BIS often serves as a common, single indicator clinicians rely upon to interpret the data acquired by a monitoring system. That is, clinicians simply rely upon the BIS indication to make clinical decisions.

As a control signal, BIS can inherently have only limited success, as the same BIS value can be produced by multiple distinct brain states. A patient under general anesthesia with isoflurane and oxygen, a patient sedated with dexmedetomidine, and a patient in stage III, or slow-wave, sleep can all have BIS values in the 40-to-60 range, which is the BIS interval in which surgery is conducted. Of these three patients, only the first is most likely in a state of “general anesthesia” and appropriate for conducting surgery. In this context, “general anesthesia” refers to unconsciousness, amnesia, analgesia, akinesia with maintenance of physiological stability. Similarly, patients anesthetized with ketamine alone or in combination with other anesthetic agents show high BIS values suggesting an awake or lightly sedated state, despite being in a state of general anesthesia. Although most reports nonetheless claim successful brain state control, such control has not been reliably demonstrated in studies involving individual subjects or real-time implementations.

Second, using BIS to account for individual variability in response to anesthetic drugs and hence, in EEG patterns, under normal, surgical, and intensive care unit conditions is a challenge. Third, EEG processing by commercially-available monitors of anesthetic state is performed, not in real-time, but with a 20-to-30-second delay. By contrast, coherence and synchrony methods provided herein, as will be described, may require only the length of time to acquire one window of data, for instance 4 seconds, followed by a short processing time much less than 1 second. Fourth, CLAD systems use ad-hoc algorithms instead of formal deterministic or stochastic control paradigms in their design. As a consequence, the reports in which CLAD systems have been implemented do not show reliable repeatable control results. Indeed, to give the appearance of successful control, the results of several subjects are often averaged in plots of CLAD performance. Finally, some have proposed the theoretical use of established control principles to design a CLAD system. However, such proposals have suggested the derivation of a wavelet-based index of anesthetic depth from the EEG, which fundamentally proposes a control signal that is analogous to BIS. Simply, until more is known about the neurophysiology of how EEG patterns relate to brain states under general anesthesia, developing generally applicable CLAD systems is a challenging problem. To this point, as described above, metrics such as BSR suffer from similar limitations and, thus, have not been suitable for developing generally applicable CLAD systems for at least the reasons discussed above.

Perhaps recognizing the complex nature of the EEG waveform and the shortcomings of BIS as a control system, Vijn and Sneyd designed CLAD systems for rats using a different metric, namely BSR, as the control signal. BSR, is defined as the proportion of time per epoch that the EEG is suppressed below a predetermined voltage threshold. The BSR ranges from 0, meaning no suppression, to 1, meaning an isoelectric EEG. The objective of such investigation was to develop a model-free approach to CLAD-system design to determine if performance of new drugs in a CLAD system could provide useful information on drug design. They processed their error signal using a non-standard deterministic control strategy that was the product of a proportional and an integral term. Although the authors claim that their CLAD system maintained control of BSR for both propofol and etomidate, they reported BSR time courses averaged over groups of rats and not for individual animals. The Vijn and Sneyd CLAD system was recently implemented by Cotten et al. to test the efficacy of new etomidate-based anesthetics in controlling BSR in rats. These authors also reported only average time courses. Accordingly there seems to be a lack of studies on the use of CLAD systems to control burst suppression in human experiments or in the ICU to maintain a level of medical coma.

To further complicate matters, there are a great number of variables that can influence the effects, effectiveness, and, associated therewith, the “level” of anesthetic influence on a given patient. Thus, closed-loop control systems can fail if the drug infusion does not account for any of the plethora of variables. Some variables include physical attributes of the patient, such as age, state of general health, height, or weight, but also less obvious variables that are extrapolated, for example, based on prior experiences of the patient when under anesthesia. When these variables are compounded with the variables of a given control system or method and the variables presented by a particular anesthetic compound or, more so, combination of anesthetic compounds, the proper and effective administration of anesthesia to a given patient can appear to be an art, rather than a science.

In addition, whether controlled by a system, such as a CLAD system, or a more traditional clinician-specific control, emergence from general anesthesia is a slow passive process achieved simply by allowing the effects of the drug to wear off. Emergence from anesthesia is traditionally a passive process whereby anesthetic drugs are merely discontinued at the end of surgery, and no drugs are administered to actively reverse their effects on the brain and central nervous system. That is, the general anesthetic agents are merely discontinued at the end of surgery, leaving the anesthesiologist and surgeon to wait for the patient to recover consciousness. The timing of emergence can be unpredictable because many factors including the nature and duration of the surgery, and the age, physical condition and body habits of the patient, can greatly affect the pharmacokinetics and pharmacodynamics of general anesthetics. Although the actions of many drugs used in anesthesiology can be pharmacologically reversed when no longer desired (e.g. muscle relaxants, opioids, benzodiazepines, and anticoagulants), this is not the case for general anesthetic induced loss of consciousness. While some basic ideas for actively reversing the effects of anesthesia have been considered, they do not translate well to traditional monitoring systems and control methods because these monitoring and control methods are generally unidirectional. For example, using burst-suppression based metrics for determining an increasing state of consciousness is counterintuitive, at best. Not surprisingly, then, control algorithms have not been developed to facilitate actively controlled recovery.

As will be described, the present disclosure overcomes drawbacks of previous technologies and provides systems and methods for monitoring and controlling a state of a patient during and after administration of an anesthetic compound or compounds.

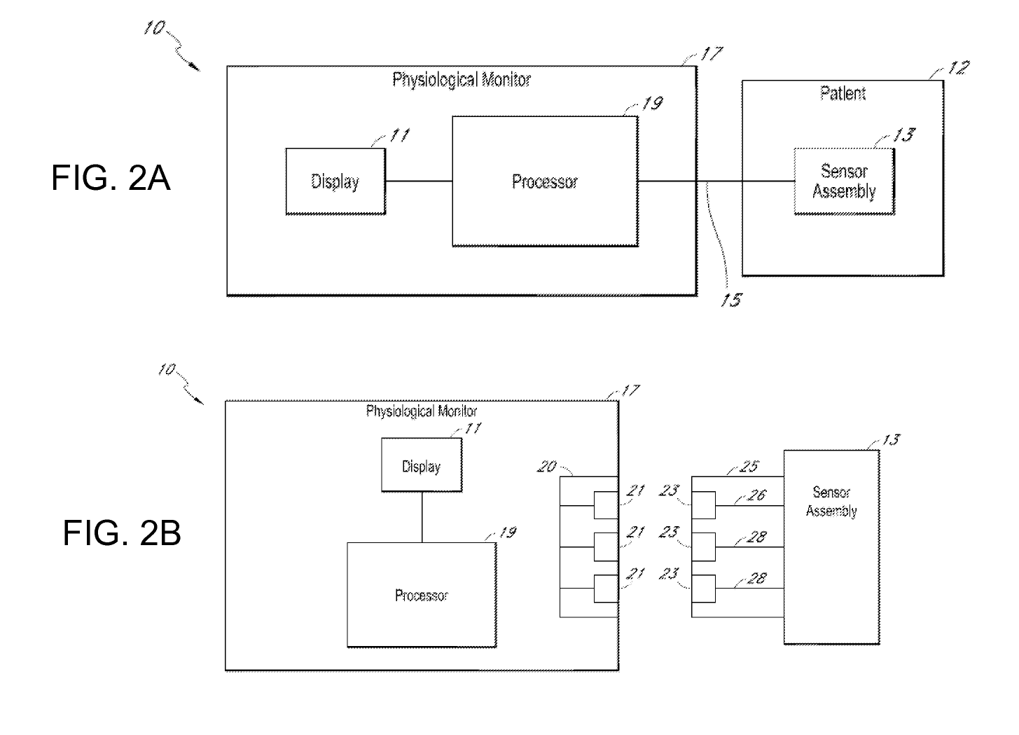

Referring specifically to the drawings, FIGS. 2A and 2B depict block diagrams of example patient monitoring systems and sensors that can be used to provide physiological monitoring and control of a patient's state, such as consciousness state monitoring, with loss of consciousness or emergence detection.

For example, FIG. 2A shows an embodiment of a physiological monitoring system 10. In the physiological monitoring system 10, a medical patient 12 is monitored using one or more sensors 13, each of which transmits a signal over a cable 15 or other communication link or medium to a physiological monitor 17. The physiological monitor 17 includes a processor 19 and, optionally, a display 11. The one or more sensors 13 include sensing elements such as, for example, electrical EEG sensors, or the like. The sensors 13 can generate respective signals by measuring a physiological parameter of the patient 12. The signals are then processed by one or more processors 19. The one or more processors 19 then communicate the processed signal to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

For clarity, a single block is used to illustrate the one or more sensors 13 shown in FIG. 2A. It should be understood that the sensor 13 shown is intended to represent one or more sensors. In an embodiment, the one or more sensors 13 include a single sensor of one of the types described below. In another embodiment, the one or more sensors 13 include at least two EEG sensors. In still another embodiment, the one or more sensors 13 include at least two EEG sensors and one or more brain oxygenation sensors, and the like. In each of the foregoing embodiments, additional sensors of different types are also optionally included. Other combinations of numbers and types of sensors are also suitable for use with the physiological monitoring system 10.

In some embodiments of the system shown in FIG. 2A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors 13.

As shown in FIG. 2B, the EEG sensor 13 can include a cable 25. The cable 25 can include three conductors within an electrical shielding. One conductor 26 can provide power to a physiological monitor 17, one conductor 28 can provide a ground signal to the physiological monitor 17, and one conductor 28 can transmit signals from the sensor 13 to the physiological monitor 17. For multiple sensors, one or more additional cables 15 can be provided.

In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor 13 and the physiological monitor 17 communicate wirelessly.

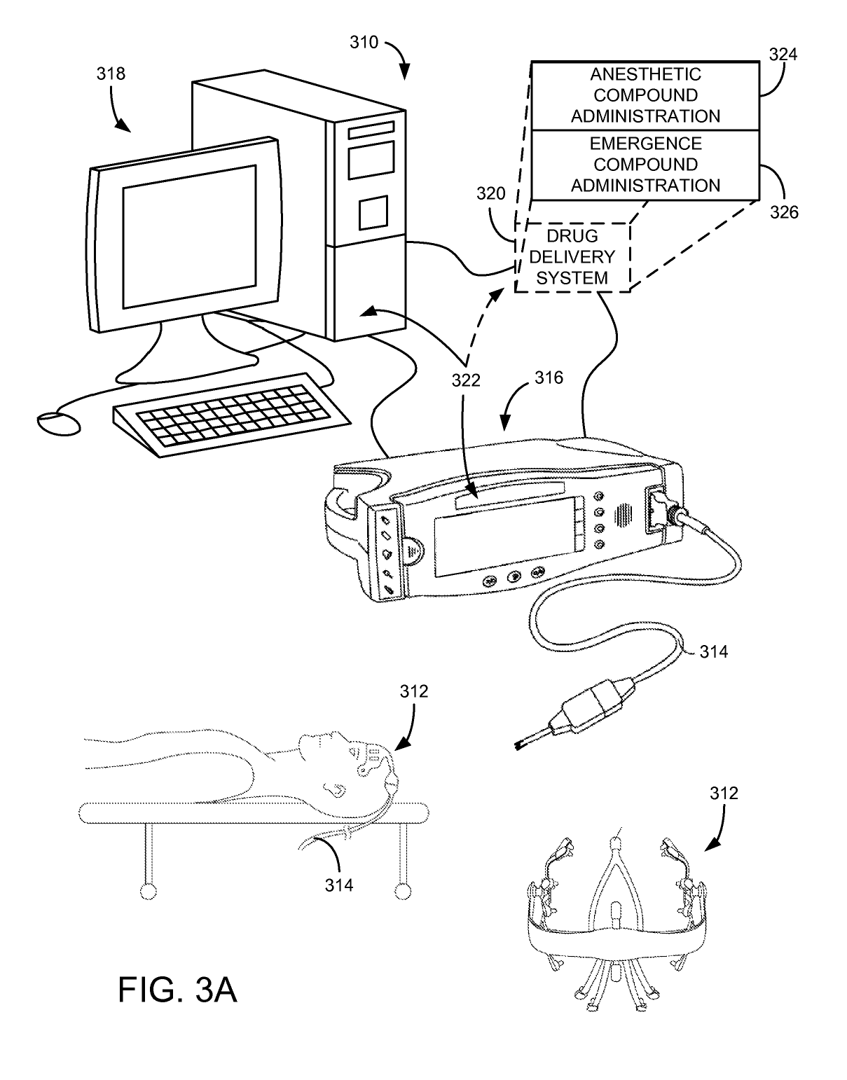

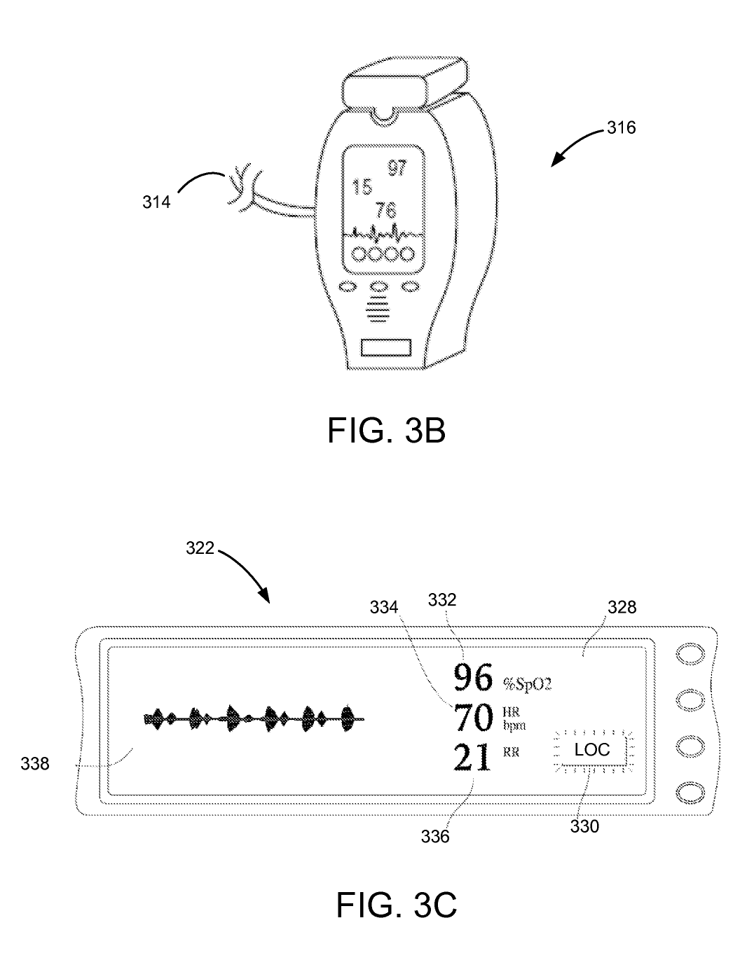

Referring now to FIG. 3A, an example system 310 for monitoring and controlling a patient during and after administration of at least one drug having anesthetic properties is illustrated. The system 310 includes a patient monitoring device 312, such as a physiological monitoring device, illustrated in FIG. 3A as an electroencephalography (EEG) electrode array. However, it is contemplated that the patient monitoring device 312 may also include mechanisms for monitoring galvanic skin response (GSR), for example, to measure arousal to external stimuli or other monitoring system such as cardiovascular monitors, including electrocardiographic and blood pressure monitors, and also ocular Microtremor monitors. One specific realization of this design utilizes a frontal Laplacian EEG electrode layout with additional electrodes to measure GSR and/or ocular microtremor. Another realization of this design incorporates a frontal array of electrodes that could be combined in post-processing to obtain any combination of electrodes found to optimally detect the EEG signatures described earlier, also with separate GSR electrodes. Another realization of this design utilizes a high-density layout sampling the entire scalp surface using between 64 to 256 sensors for the purpose of source localization, also with separate GSR electrodes.

The patient monitoring device 312 may be connected via a cable 314 to communicate with a monitoring system 316, which may be a portable system or device (as shown in FIG. 3B), and provides input of physiological data acquired from a patient to the monitoring system 316. Also, the cable 314 and similar connections can be replaced by wireless connections between components. As illustrated in FIG. 3A, the monitoring system 316 may be further connected to a dedicated analysis system 318. Also, the monitoring system 316 and analysis system 318 may be integrated.

The monitoring system 316 may be configured to receive raw signals acquired by the EEG electrode array and assemble, and even display, the raw signals as EEG waveforms. Accordingly, the analysis system 318 may receive the EEG waveforms from the monitoring system 316 and, as will be described, analyze the EEG waveforms and signatures therein based on a selected anesthesia compound, determine a state of the patient based on the analyzed EEG waveforms and signatures, and generate a report, for example, as a printed report or, preferably, a real-time display of signature information and determined state. However, it is also contemplated that the functions of monitoring system 316 and analysis system 318 may be combined into a common system. In one aspect, the monitoring system 316 and analysis system 318 may be configured to determine, based on measures of brain coherence and synchrony, a current and future brain state under administration of anesthetic compounds, such as during general anesthesia or sedation.

In some configurations, the system 310 may also include a drug delivery system 320. The drug delivery system 320 may be coupled to the analysis system 318 and monitoring system 316, such that the system 310 forms a closed-loop monitoring and control system. Such a monitoring and control system in accordance with the present disclosure is capable of a wide range of operation, but includes user interfaces 322 to allow a user to provide any input or indications to configure the monitoring and control system, receive feedback from the monitoring and control system, and, if needed reconfigure and/or override the monitoring and control system.

Referring specifically to FIG. 3C, a non-limiting example of a user interface 322 for a monitoring system 316 is illustrated, which may include a multiparameter physiological monitor display 328. For example, the display 328 can output a loss of consciousness (“LOC”) indicator 330. The loss of consciousness indicator 330 can be generated using any of the techniques, as described. The display 328 may also provide parameter data using an oxygen saturation (“SpO₂”) indicator 332, a pulse rate indicator 334, and a respiration rate indicator 336, any other indicator representative of any desired information. In the depicted embodiment shown in FIG. 3C, the LOC indicator 330 includes text that indicates that the patient has lost consciousness. In some embodiments, the LOC indicator 330 may include an index indicating a state of consciousness, of the patient. The text displayed in the LOC indicator 330 may depend on a confidence calculation from one of the consciousness state detection processes described above. Each one of the consciousness state detection processes described above may have different confidence rating depending on how accurately the particular process or combination of processes can predict a state of consciousness condition. The confidence rating may be stored in the patient monitor. In some embodiments, more than one of processes (described above) can be used to determine the LOC indicator 330. Furthermore, the display 328 can also provide any segment of raw or processed waveform signals 338 as output, including time-series EEG signals, intermittently or in real time.

Referring back to FIG. 3A, in some configurations, the drug delivery system 320 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness.

For example, in accordance with one aspect of the present disclosure, methylphenidate (MPH) can be used as an inhibitor of dopamine and norepinephrine reuptake transporters and actively induces emergence from isoflurane general anesthesia. MPH can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. Also, ethylphenidate or other agents can be used to actively induce emergence from isoflurane, propofol, or other general anesthesia by increasing arousal using a control system, such as described above.

Therefore, a system, such as described above with respect to FIG. 3A, can be provided to carry out active emergence from anesthesia by including a drug delivery system 320 with two specific sub-systems. As such, the drug delivery system 320 may include an anesthetic compound administration system 324 that is designed to deliver doses of one or more anesthetic compounds to a subject and may also include a emergence compound administration system 326 that is designed to deliver doses of one or more compounds that will reverse general anesthesia or enhance the natural emergence of a subject from anesthesia.

For example, MPH and analogues and derivatives thereof induces emergence of a subject from anesthesia-induced unconsciousness by increasing arousal and respiratory drive. Thus, the emergence compound administration system 326 can be used to deliver MPH, amphetamine, modafinil, amantadine, or caffeine to reverse general anesthetic-induced unconsciousness and respiratory depression at the end of surgery. The MPH may be dextro-methylphenidate (D-MPH), racemic methylphenidate, or leva-methylphenidate (L-MPH), or may be compositions in equal or different ratios, such as about 50%:50%, or about 60%:40%, or about 70%:30%, or 80%:20%, 90%:10%, 95%:5% and the like. Other agents may be administered as a higher dose of methylphenidate than the dose used for the treatment of Attention Deficit Disorder (ADD) or Attention Deficit Hyperactivity Disorder (ADHD), such as a dose of methylphenidate can be between about 10 mg/kg and about 5 mg/kg, and any integer between about 5 mg/kg and 10 mg/kg. In some situations, the dose is between about 7 mg/kg and about O0.1 mg/kg, or between about 5 mg/kg and about 0.5 mg/kg. Other agents may include those that are inhaled.

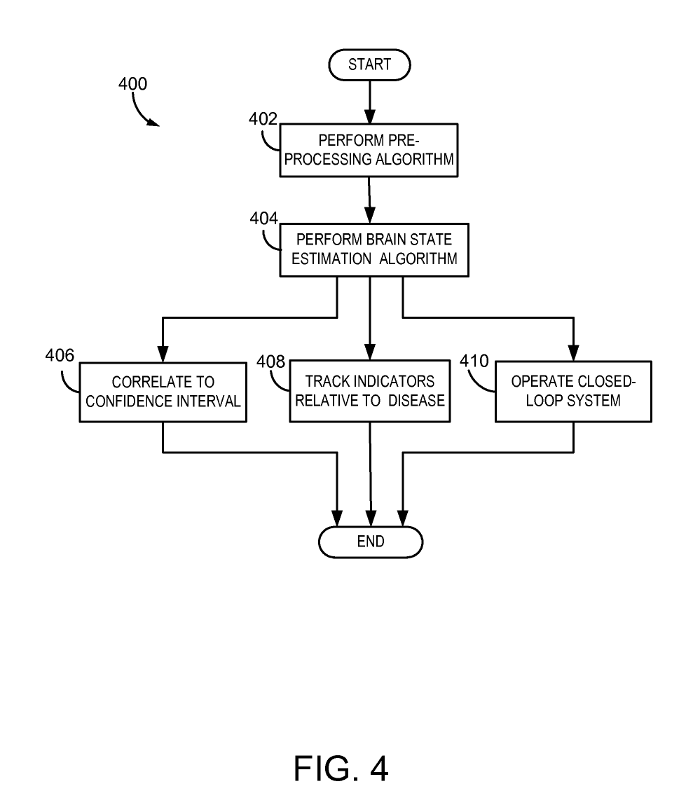

Turning now to FIG. 4, a process 400 for monitoring and controlling a state of a patient in accordance with the present disclosure begins at process block 402 by performing a pre-processing algorithm that analyzes waveforms acquired from an EEG monitoring system, as described. In some aspects, at process block 402, indicators related to the EEG waveforms may be identified, such as spike rates, burst suppression rates, oscillations (for example, slow or low-frequency oscillations in the range between 0.1 and 1 Hz), power spectra characteristics, phase modulations, and so forth. At this step, raw EEG waveforms may be modified, transformed, enhanced, filtered, or manipulated to take any desired or required form, or possess any desired or required features or characteristics. The pre-processed data is then, at process block 404, provided as an input into a brain state estimation algorithm. In one aspect, the brain state estimation algorithm may perform a determination of current and/or future brain states related to measures of brain synchrony and/or coherence, under administration of any combination of anesthetic compounds, such as during general anesthesia or sedation.

The brain state estimation algorithm output, at process block 406, may be correlated with “confidence intervals.” The confidence intervals are predicated on formal statistical comparisons between the brain state estimated at any two time points. Also, at process block 408, the output of the brain state estimation algorithm can be used to identify and track brain state indicators, such as spike rates, low-frequency oscillations, power spectra characteristics, phase modulations, and so forth, during medical procedures or disease states. Exemplary medically-significant states include hypothermia, general anesthesia, medical coma, and sedation to name but a few. The output of the brain state estimation algorithm may also be used, at process block 410 as part of a closed-loop anesthesia control process.

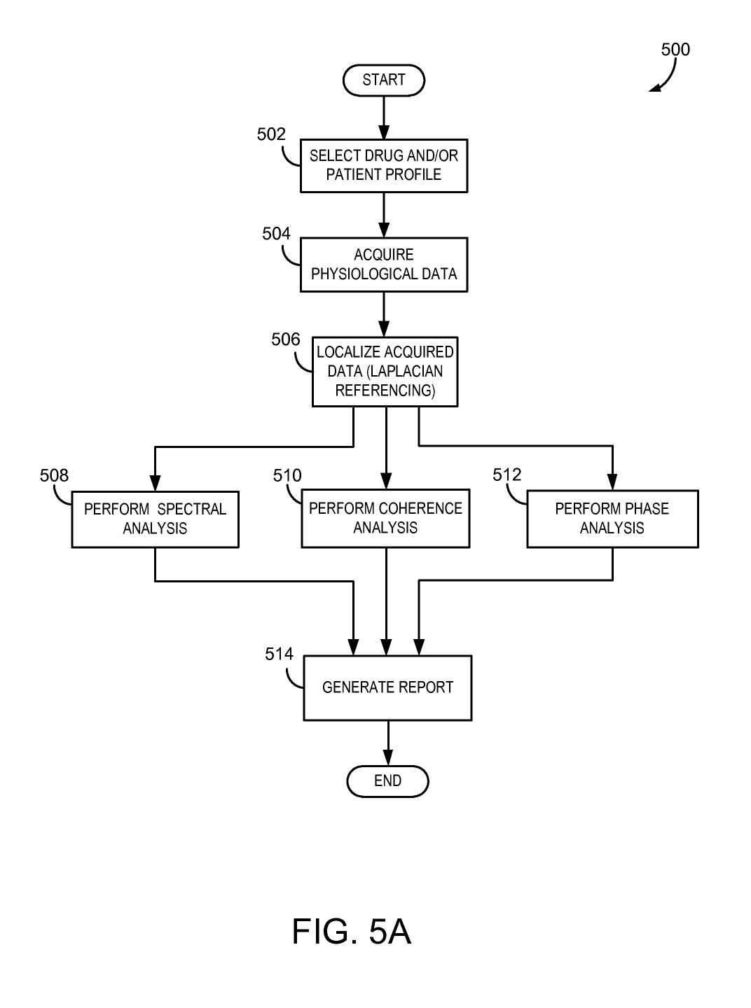

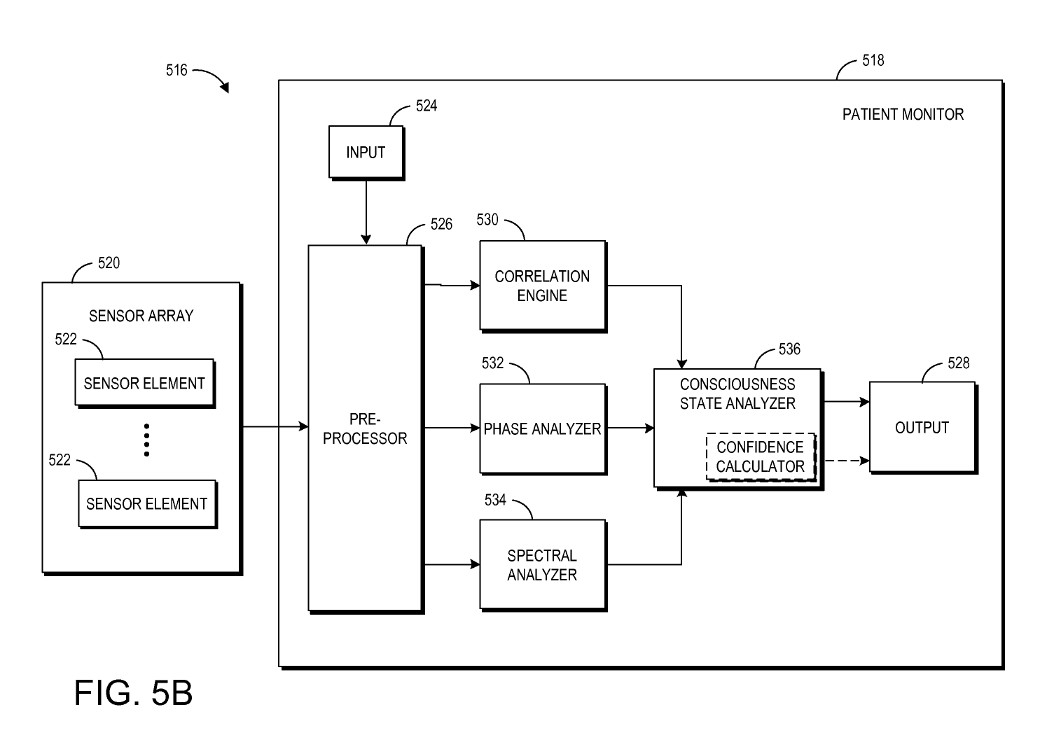

Also, the present disclosure provides methods for determining a brain state of a patient, using systems as described. Referring now to FIG. 5, a process 500 begins at process block 502 with the selection of a desired drug, such as anesthesia compound or compounds, and/or an indication related to a particular patient profile, such as a patient's age, height, weight, gender, or the like. Furthermore, drug administration information, such as timing, dose, rate, and the like, in conjunction with the above-described EEG data may be acquired and used to estimate and predict future patient states in accordance with the present disclosure. As will be described, the present disclosure recognizes that the physiological responses to anesthesia vary based on the specific compound or compounds administered, as well as the patient profile. For example, elderly patients have a tendency to show lower amplitude alpha power under anesthesia, with some showing no visible alpha power in the unconscious state. The present disclosure accounts for this variation between an elderly patient and a younger patient. Furthermore, the present disclosure recognizes that analyzing physiological data for signatures particular to a specific anesthetic compound or compounds administered and/or the profile of the patient substantially increases the ability to identify particular indicators of the patient's brain being in a particular state and the accuracy of state indicators and predictions based on those indicators.

For example, the following drugs are examples of drugs or anesthetic compounds that may be used with the present disclosure: Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and the like. However, the present disclosure recognizes that each of these drugs, induces very different characteristics or signatures, for example, within EEG data or waveforms.

With the proper drug or drugs and/or patient profile selected, acquisition of physiological data begins at process block 504, where the acquired data is EEG data. The present disclosure provides systems and methods for analyzing acquired physiological information from a patient, analyzing the information and the key indicators included therein, and extrapolating information regarding a current and/or predicted future state of the patient. To do so, rather than evaluate physiological data in the abstract, the physiological data is processed. Processing can be done in the electrode or sensor space or extrapolated to the locations in the brain. As will be described, the present disclosure enables the tracking of the spatiotemporal dynamics of the brain by combining additional analysis tools, including, for example, spectrogram, phase-amplitude modulation, coherence analyses, and so forth. As will be apparent, reference to “spectrogram” may refer to a visual representation of frequency domain information.

At process block 506, Laplacian referencing can be performed to estimate radial current densities perpendicular to the scalp at each electrode site of, for example, the monitoring device of FIG. 3A. This may be achieved by taking a difference between voltages recorded at an electrode site and an average of the voltage recorded at the electrode sites in a local neighborhood. Other combinations of information across the plurality of electrodes may also be used to enhance estimation of relevant brain states. In this manner, generated signals may be directly related to electrodes placed on a subject at particular sites, such as frontal, temporal, parietal locations, and so forth, or may be the result of combinations of signals obtained from multiple sites.

Next, at process blocks 508, 510, 512, different analyses may be performed either independently, or in any combination, to yield any of spectral, temporal, coherence, synchrony, amplitude, or phase information, related to different spatiotemporal activities at different states of a patient receiving anesthesia. In some aspects, information related to brain coherence and synchrony may be determined in relation to slow or low-frequency oscillations.

At process block 508, a spectral analysis may performed to yield information related to the time variation of spectral power for signals assembled from physiological data acquired at process block 504. Such spectral analysis may facilitate identification and quantification of EEG signal profiles in a target range of frequencies. In some aspects, spectrograms may be generated and processed at process block 508, for example, using multitaper and sliding window methods to achieve precise and specific time-frequency resolution and efficiency, which are properties that can be used to estimate relevant brain states. In other aspects, state-space models of dynamic spectra may be applied to determine the spectrograms, whereby the data drives the optimal amount of smoothing. Although spectrogram generation and processing may be performed at process block 508, a visual representation of the spectrograms need not be displayed.

At process block 510, a coherence analysis may be performed to give indications related to spatial coherence across local and global brain regions, using signals generated from raw or processed physiological data, as described. In particular, coherence quantifies the degree of correlation between any pair signals at a given frequency, and is equivalent to a correlation coefficient indexed by frequency. For example, a coherence of 1 indicates that two signals are perfectly correlated at that frequency, while a coherence of 0 indicates that the two signals are uncorrelated at that frequency. In some aspects, coherence may determined for signals described by specific frequency bands, such as low or slow oscillation frequencies (for example, 0.1-1 Hz), or δ (1-4 Hz), α (8-14 Hz), γ (20-40 Hz) frequency bands and so forth, identified by way of a spectral analysis, as performed at process block 508. For example, a strong coherence in the a range indicates highly coordinated activity in the frontal electrode sites.

Other features of generated signals, as described, may likewise be tracked, such as phase-amplitude and phase-phase modulations. Thus, at process block 512, a phase analysis may be performed that considers the amplitude or phase of a given signal with respect to the amplitude or phase of other signals. In particular, as explained above, spectral analysis of EEG signals allows the present disclosure to track systematic changes in the power in specific frequency bands associated with administration of anesthesia, including changes in slow or low frequencies (0.1-1 Hz), δ (1-4 Hz), θ (5-8 Hz), α (8-14 Hz), β (12-30 Hz), and γ (30-80 Hz). However, spectral analysis treats oscillations within each frequency band independently, ignoring correlations in either phase or amplitude between rhythms at different frequencies. In some aspects, computations related to the extent that slow or low-frequency oscillation phases modulate the amplitudes of oscillations in other frequency bands, or spiking activity may be performed. In other aspects, phase relationships between signals, such as slow-oscillation signals, from different cortical regions may also be determined to provide synchrony information in relation to different states of a patient receiving anesthesia.

The above-described selection of an appropriate analysis context based on a selected drug or drugs (process block 502), the acquisition of data (process block 504), and the analysis of the acquired data (process blocks 508-512) set the stage for the new and substantially improved real-time analysis and reporting on the state of a patient's brain as an anesthetic or combination of anesthetics is being administered and the recovery from the administered anesthetic or combination of anesthetics occurs. That is, although, as explained above, particular indications or signatures related to the states of effectiveness of an administered anesthetic compound or anesthetic compounds can be determined from each of the above-described analyses (particularly, when adjusted for a particular selected drug or drugs), the present disclosure provides a mechanism for considering each of these separate pieces of data and more to accurately indicate and/or report on a state of the patient under anesthesia and/or the indicators or signatures that indicate and may be used to control the state of the patient under anesthesia.

Specifically, referring to process block 514, any and all of the above-described analysis and/or results can be combined and reported, in any desired or required shape or form, including providing a report in real time, and, in addition, can be coupled with a precise statistical characterizations of behavioral dynamics, for use by a clinician or use in combination with a closed-loop system as described above. In some aspects, information related to brain coherence and synchrony may be employed. In particular, behavioral dynamics, such as the points of loss-of-consciousness and recovery-of-consciousness can be precisely, and statistically calculated and indicated in accordance with the present disclosure. To do so, the present disclosure may use dynamic Bayesian methods that allow accurate alignment of the spectral, coherence and phase analyses relative to behavioral markers.

Referring to FIG. 5B, a system 516 for carrying out steps for determining a brain state of a patient, as described above, is illustrated. The system 516 includes patient monitor 518 and a sensor array 520 configured with any number of sensors 522 designed to acquire physiological data, such as EEG data. The sensor array 520 is in communication with the patient monitor 518 via a wired or wireless connection.

The patient monitor 518 is configured to receive and process data provided by the sensor array 522, and includes an input 524, a pre-processor a processor 526 and an output 528. In particular, the pre-processor 526 is configured to carry out any number of pre-processing steps, such as assembling the received physiological data into time-series signals and performing a noise rejection step to filter any interfering signals associated with the acquired physiological data. The pre-processor is also configured to receive an indication via the input 524, such as information related to administration of an anesthesia compound or compounds, and/or an indication related to a particular patient profile, such as a patient's age, height, weight, gender, or the like, as well as drug administration information, such as timing, dose, rate, and the like. The patient monitor 518 further includes a number of processing modules in communication with the pre-processor 526, including a correlation engine 530, a phase analyzer 532 and a spectral analyzer 534. The processing modules are configured to receive pre-processed data from the pre-processor 526 and carry out steps necessary for determining a brain state of a patient, as described, which may be performed in parallel, in succession or in combination. Furthermore, the patient monitor 518 includes a consciousness state analyzer 536 which is configured to received processed information, such as coherence and synchrony information, from the processing modules and provide a determination related to a present or future state of a patient under anesthesia and confidence with respect to the determined state(s). Information related to the determined state(s) may then be relayed to the output 528, along with any other desired information, in any shape or form. For example, the output 528 may include a display configured to provide a consciousness indicator and confidence indicator, either intermittently or in real time.

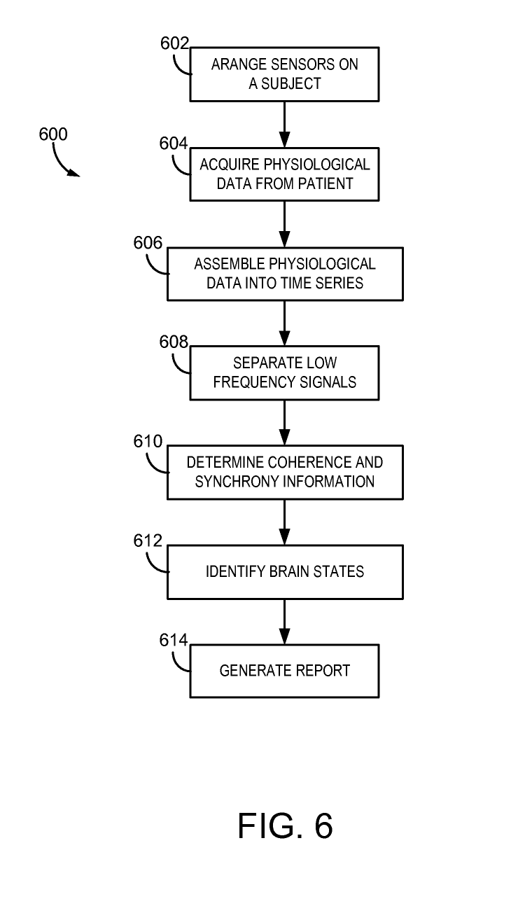

Turning now to FIG. 6, a flow chart is illustrated setting forth steps of a method for monitoring a patient in accordance with the present disclosure. The process 600 begins at process block 602, whereby any number of sensors may be arranged on a subject, and a clinician or operator may provide at least one indication related to the administration of a drug, or a patient characteristic. Then, at process block 604, any amount of physiological data may be acquired, which may then, at process block 606, be arranged into time-series data. Subsequently, at process block 608, low frequency signals may be separated from the time-series data, using any frequency dependent approaches, such as band-pass, or low-pass filtering. Such signals may, in some aspects, be representative of a frequency range between 0.1 Hz and 1 Hz. Using at least indicators from such low frequency signals, at least one of a coherence or synchrony information may be generated. Such information may provide spatiotemporal signatures, as described, which, when employed in association with a model, may identify brain states at process block 612, including at least one of a current and future state, consistent with the administration of at least one drug having anesthetic properties. Finally, at process block 614, a report may be generated, taking any shape or form, as desired or required. Such report may provide an indication to a clinician regarding at least one of a current and future brain state.

The above-described systems and methods may be further understood by way of examples. These examples are offered for illustrative purposes only, and are not intended to limit the scope of the present disclosure in any way. Indeed, various modifications of the disclosure in addition to those shown and described herein will become apparent to those skilled in the art from the foregoing description and the following examples and fall within the scope of the appended claims. For example, specific examples of brain states, medical conditions, levels of anesthesia or sedation and so on, in association with specific drugs and medical procedures are provided, although it will be appreciated that other drugs, doses, states, conditions and procedures, may be considered within the scope of the present disclosure. Furthermore, examples are given with respect to specific indicators related to brain states, although it may be understood that other indicators and combinations thereof may also be considered within the scope of the present disclosure. Likewise, specific process parameters are recited that may be altered or varied based on variables such as signal amplitude, phase, frequency, duration and so forth.

### Example I

General anesthesia, a drug-induced reversible coma, is commonly initiated by administering a large dose of a fast-acting drug, such as propofol, to induce unconsciousness within seconds. This state that may be maintained as long as needed to execute surgical and many nonsurgical procedures. Although much is known about molecular actions of anesthetics, it is not clear how these effects at molecular targets affect single neurons and larger-scale neural circuits to produce unconsciousness.

The macroscopic dynamics of anesthetics are noticeable in EEGs, which contain several stereotyped features. For example, when patients are awake, corresponding spectrograms show strong occipital so-called a activity, while after loss of consciousness using propofol, the spectrograms show loss of a activity and increased δ activity in the occipital sites, with strong α and δ activity in the frontal sites. Increased power in frontal sites over the α (8-14 Hz), β (12-30 Hz), and δ (1-4 Hz) ranges occurring after loss of consciousness is consistent with previously observed pattern of anteriorization, and as patients lose responsiveness, the coordinated activity over the occipital sites in the α range diminishes. When patients are unconscious, strong coordinated activity in the α range is observed broadly over the frontal electrode sites at which the spectrograms show the anteriorization pattern. Despite the overall high δ activity in the spectrograms, coordinated activity may only be observed in the α range. The relative power in the occipital α and δ ranges tracks the patients' behavioral responses, whereby the occipital α power is greater than the δ power when the patient is awake, and the reverse is true when the patients are unconscious. Also, in the case of general anesthesia maintenance using propofol, spectrograms showing increased δ and γ power, also show a coherent α (˜10 Hz) rhythm across frontal cortex along with burst suppression and slow oscillations (<1 Hz).

Although many such patterns are observed consistently, it is unclear how they are functionally related. For example, a transition to unconsciousness can occur in tens of seconds in the case of general anesthesia, but many neurophysiological features continue to fluctuate for minutes after induction and are highly variable between different levels of general anesthesia. In addition, the dynamic interactions between cortical areas that underlie the EEG oscillations are not well understood, because few studies have simultaneously recorded ensembles of single neurons and oscillatory dynamics from sites distributed throughout the brain.

As such, both neuronal and circuit-level dynamics were investigated in the human brain during induction of unconsciousness with propofol. Simultaneous recordings were obtained from single units, local field potentials (LFPs), and intra-cranial electrocorticograms (ECoG) over up to 8 cm of cortex, enabling examination of neural dynamics at multiple spatial scales with millisecond scale temporal resolution. The spatial and temporal organization of neural dynamics during the evolution of unconsciousness was investigated to identify mechanisms associated with cortical integration.

### Methods

### Data Collection

Three patients with epilepsy intractable to medication were implanted with intracranial subdural electrocorticography electrodes for standard clinical monitoring (AdTech). In some instances, ECoG electrode placement was determined by clinical criteria, and the electrodes were located in temporal, frontal, and parietal cortices. Individual ECoG electrodes within a grid were spaced 1 cm apart. In addition, a 96-channel NeuroPort microelectrode array with 1.0-mm-long electrodes (BlackRock Microsystems) was implanted into the superior (patient B) or middle (patients A and C) temporal gyrus to record LFPs and ensembles of single units for research purposes. In each patient, the Neuroport array was located at least 2 cm from the seizure focus. All recordings were collected at the beginning of surgery to explant the electrodes. Anesthesia was administered as a bolus dose of propofol according to standard clinical protocol. All propofol doses were based on the anesthesiologist's clinical judgment rather than the research study considerations. Patient A received three boluses (130 mg, 50 mg, and 20 mg), patient B received one bolus (200 mg), and patient C received one (150 mg). After induction, patients were transferred to a continuous intravenous infusion of propofol to maintain anesthetic levels.

Throughout the induction period, patients responded to auditory stimuli (prerecorded words and the patient's name) with a button press, and stimuli were presented every 4 s to obtain precision for LOC (loss of consciousness) time on the order of seconds. LOC time was defined as the period from −1 to 4 s surrounding the first stimulus after the patient completely ceased responding. Spike sorting was carried out with Offline Sorter (Plexon) and produced 198 single units for further analysis. LFPs were referenced to a wire distant from the microelectrode array and were collected with hardware filters band-passing between 0.3-7,500 Hz with a sampling rate of 30 kHz. LFPs then were low-pass filtered at 100 Hz and re-sampled to 250 Hz. For display, raw time-series were low-pass-filtered with a finite-impulse response filter with 4,464 coefficients, achieving unit gain between 0 and 40 Hz and attenuation of more than −300 dB above 42 Hz.

ECoG recordings were collected with a sampling rate of either 250 Hz (patients B and C) or 2,000 Hz (patient A), in which case it was low-pass-filtered at 100 Hz and re-sampled to 250 Hz. ECoG recordings were referenced to an intracranial reference strip channel when available (patient A) and otherwise to an average reference. In patients A and B, ECoG recordings were collected throughout. In patient C, the microelectrode recordings were collected throughout, but the ECoG recording ended ˜100 s after LOC; therefore the significance of slow oscillation phase-coupling could not be assessed in ECoG channels, because the spike rate was nearly zero during this time. Two ECoG grid channels were rejected in patient A because of large artifacts. All data were exported to Matlab (Mathworks) for analysis with custom software.

### Spike Rate Analysis

Spike rates and confidence intervals were computed with Bayesian state-space estimation. To minimize any error caused by unstable recordings, the spike rate analysis excluded units that were not confidently detected throughout the entire baseline period (8.1%). The computed spike-rate effects were similar when these units were included. Periods of silence were compared with a simulated Poisson distribution of equal rate over each 10-s period, and significance was assessed for each patient with a X² test relative to that distribution.

### Spectral Analysis

Spectrograms were calculated with multitaper methods using the Chronux toolbox (http://chronux.org/). Power changes after LOC were computed as the percent change in the period 30-60 s after LOC relative to the period 30-60 s before LOC. Slow oscillations were extracted by applying a symmetric finite impulse-response band-pass filter with 4,464 coefficients, achieving unit gain from 0.1-1 Hz and attenuation of more than −50 dB from 0-0.85 and 1.15-125 Hz. Because of hardware filter settings with a high pass at 0.3 Hz, the power contribution below 0.3 Hz was minimized. Phase was extracted with a Hilbert transform. Statistical testing of triggered spectrograms was done by taking a ratio of each X² distribution, and significance was calculated as an F-test with a Bonferroni correction for multiple comparisons across frequencies. For comparing spectra during and before an ON period, power spectra from 250 ms after ON period onset were compared with spectra from 250 ms before the ON period onset. Averaged LFP waveforms were compared by preselecting a time period and performing a t-test on the mean amplitude values within that interval. When comparing the waveform height before and after spiking, a t-test was performed comparing the mean amplitude in the time window from −750 to 500 ms and in the time window from 500-750 ms locked to ON period onset or slow oscillation minimum.

### Phase Modulation

Significance for single-unit phase-coupling was computed with a X² test on the binned phase distribution. The analysis was performed a second time on only cells with spike rates above 0.1 Hz, ensuring that there were at least five expected spikes per phase bin. Strength of phase modulation was computed using a modulation index (MI) adapted to quantify the Kullback-Liebler divergence of the phase histogram from the uniform distribution, measured in bits. Spike phase was split into a phase histogram (p) of 10 bins, and MI was computed as Σᵢ₌₁¹⁰pᵢ log₂pᵢ+log₂10. The X² statistic was also computed as an alternative measure, yielding similar results. MI significance for each ECoG channel was calculated by shuffling the entire spike train randomly between 2 and 10 s and calculating a shuffled MI over 2,000 random shifts. The empirical MI then was compared with the shuffled MI with a significance level of 0.05 and a Bonferroni correction for multiple comparisons across channels. For LFP phase analysis, each single unit was compared with its local LFP channel. The time-varying phase modulation was computed with a window of 20 s sliding every 5 s. To assess the phase of maximal spiking relative to the ECoG slow oscillations, the phase of spiking was divided into 20 bins, and then the mode of the phase histogram was reported.

### Timing of Spike Rate and Spectral Power Changes Relative to LOC

Spike rates and spectral power were tested to determine the first time bin in which these features differed significantly from the baseline period before propofol administration. Every time point was compared, starting 30 s before LOC, with a baseline of spike rates or spectral features from the 3-min baseline period immediately preceding it. To assess spike rate significance, a Bayesian hierarchical analysis was used in which each post baseline time point was compared with samples drawn from the Gaussian distribution of the baseline period and tested for a significant difference. This baseline sampling distribution was computed with the same state-space algorithm used to calculate spike rates. To determine the time at which power at a given frequency differed significantly, an analogous method was used where the Gaussian sampling distribution replaced with a X² distribution, which is the appropriate distribution for power measures. The baseline was not re-sampled and instead the time at which MI became higher than the mean of the baseline period plus two SDs was reported. For all these measures, 5-s non-overlapping bins were used to identify the time at which changes occurred relative to LOC, which is the period from −2.5 to 2.5 s.

### Phase Locking Factor

The PLF was computed to obtain a time-varying measure of phase offsets between slow oscillations. The phase of the slow oscillation was extracted as described in Spectral Analysis. For each time point, the quantity z(t)=exp{−i*[φ_{A}(t)−φ_{B}(t)]} was computed, where φ_{A}(t) is the phase of one ECoG slow oscillation at each time point and φ_{B}(t) is the phase of another ECoG slow oscillation. The PLF then was calculated as the mean of z(t) across the pre-LOC periods and across the post-LOC period. To assess the variability of phase offsets, the magnitude of the PLF was calculated. The distribution of PLF magnitude was assessed by plotting the mean and SD of the PLF magnitude across each pair of ECoG channels separated by a given distance (the distance between channels computed geometrically across the grid). To determine the mean value of the phase offset across time, the angle of the PLF was calculated. The distribution of mean phase offsets across all pairs of channels separated by a given distance was then plotted by taking a 2D histogram of PLF angle values for all electrode pairs. Accompanying reconstructed brain showed individually localized electrode positions. The PLF provides the same information as the coherence, (described below), estimating the same underlying quantity, but with a different estimation method.

### GLM Fitting

A GLM was fit to ensemble spiking using custom software that performed regression with Truncated Regularized Iteratively Reweighted Least Squares (TR-IRLS) (26, 50) and using the Bayesian Information Criterion to select the best model. Using the Akaike's Information Criterion also yielded a significant contribution of spike history. The GLM was constructed to predict ensemble spiking, which was defined as a series of 12-ms bins that contained a 1 if any spikes from any units occurred in that period and a 0 otherwise. Ten covariates were used to represent the range of possible LFP phase values. Amplitude was normalized to range between 0 and 1. Because individual unit spike rates are low, the history-dependent terms in this model predominately reflect interactions between units. The version presented is with 12-ms bins of spike history; similar results were obtained when using 4- or 8-ms bins. We excluded the minute surrounding LOC to ensure that any correlation between pre-LOC and post-LOC analyses did not result from bias from adjacent recordings during the LOC transition.

### Single-Unit Correlations

Single units with high post-LOC spike rates were selected for correlation analysis to ensure sufficient spikes to assess the significance of their correlations. The minute surrounding LOC was excluded to reduce bias that could result from comparing adjacent recordings. Correlations between single units were computed relative to a shuffled baseline, to examine fine time-scale synchronization beyond the changes in population spike rate induced by the slow oscillation. Spike times were randomly shuffled 200 times, between 50 and 500 ms, to obtain a baseline of correlated spike rate without millisecond-level timing information. Paired correlations then were tested for significance between −100 and 100 ms, with P<0.05 using a Bonferroni correction for multiple comparisons across lags. Correlations were judged significant if they had a P<0.05 departure from the Poisson distribution of spike occurrence predicted by the shuffled baseline. The relationship between pairs of single units was visualized with the square root of the estimate of the cross-intensity function. Fisher's exact test was performed in R statistical software (http://www.r-project.org/).

### Detecting Initiation of ON Periods

ON periods were detected by binning spikes from all units in 50-ms time bins and then setting a threshold to detect local peaks in the spike rate. The threshold was determined manually for each patient after visually checking to ensure adequate detection, because the number of units and thus expected population spike rates differed in each patient. After detection, the first spike within 300 ms of ON period detection was taken as the initiation time, and spike histograms verified that these times represented initiation of spiking. These ON period initiation times then were used for subsequent analysis of slow oscillation spectra and waveform morphology.

### Results

We recorded single units (n=198), LFPs, and ECoG in three patients undergoing intracranial monitoring for surgical treatment of epilepsy. Single units and LFPs were recorded from a 96-channel microelectrode array implanted in temporal cortex for research purposes. We recorded throughout induction of general anesthesia by bolus administration of propofol before planned neurosurgery to remove the electrodes. Patients performed an auditory task requiring a button press in response to stimuli. All patients completely ceased responding to the task within 40 s of propofol administration and remained unresponsive for the remainder of the recording period, lasting 5-10 min after LOC. LOC was defined as the onset of this period of unresponsiveness to auditory stimuli. To acknowledge the fact that LOC could have occurred at any point between the last response and the failure to make the next response, LOC was defined as the interval beginning 1 s before the first missed stimulus up until the second missed stimulus (5 s total). We then compared spectra across all ECoG channels in the pre- and post-LOC periods and found that average spectra in the post-LOC period differed significantly from those in the pre-LOC period: Slow (0.1-1 Hz) and gamma (25-40 Hz) power increased in the unconscious state. These results suggest that propofol did not reveal any gross disruption of GABA networks.

### Spike Rates are Highly Variable After LOC

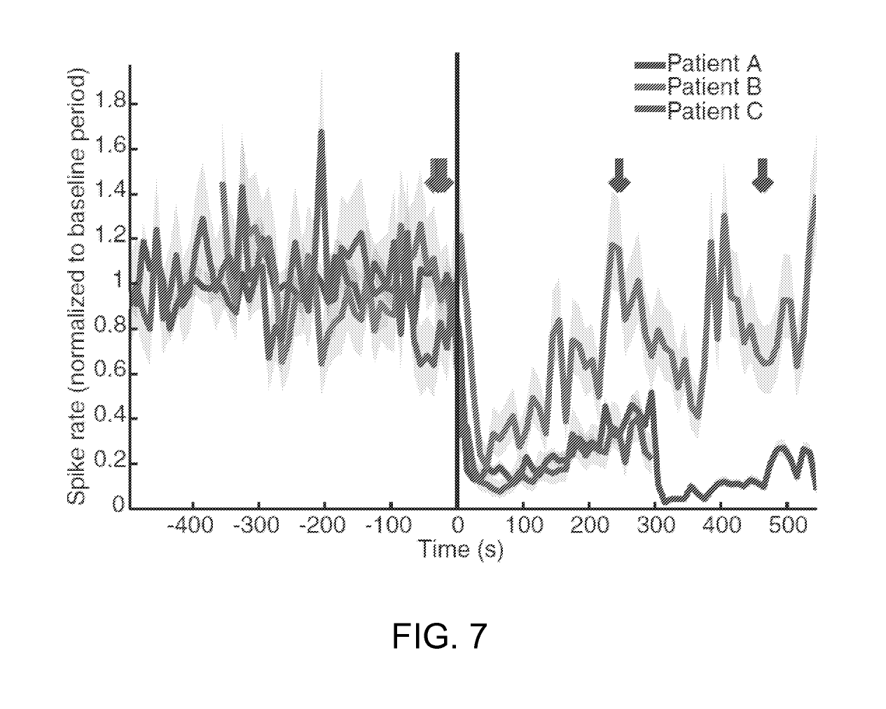

To determine the relationship between changes in spike rate and LOC, we first examined the overall spike rate in a local network of cortical neurons. Consistent with propofol's enhancement of GABA-ergic signaling, widespread suppression of spiking was observed after LOC. In each patient, the spike rate across the population of units decreased significantly 0-30 s after LOC, shown in FIG. 7. Mean spike rates across all units reached a minimum 35-85 s after LOC, having decreased 81-92% from the baseline awake state. However, spike rates subsequently recovered over several minutes. At 4 min after LOC, the rate across the entire population of units varied widely, ranging from 33% of baseline in patient A to 117% of baseline in patient B. At this 4-min post-LOC period, individual units also displayed a wide range of spike rates, with some as high as or higher than baseline; only 35.2% of units still had spike rates significantly below baseline, 55.1% of units were not significantly different, and 9.7% of units had significantly increased spike rates. We conclude that propofol rapidly causes a nearly complete but transient suppression of cortical spiking, and after several minutes many individual neurons recover to baseline spike rates. The fluctuation in spike rates across time, which could have come about from changing propofol blood levels, demonstrates that brain state is dynamic after LOC. However, subjects remained unconscious throughout this period despite widely varying spike rates, suggesting that unconsciousness is not strictly associated with gross changes in spike rate.

Spiking Activity is Organized into Periods of Activity and Quiescence After LOC

Given that mean spike rates did not exhibit a fixed relationship with state of consciousness, we examined whether unconsciousness was associated instead with a change in the temporal structure of spiking. We observed that spiking activity across the population of units occurred in short periods of activity that were interrupted by periods of silence. To estimate conservatively the amount of time with no spike activity, we binned spikes from all units into 400-ms bins. We found that 63% of bins contained no spikes, significantly more than simulated neurons with a constant rate (33%, P<0.001 for each patient, Pearson's X² test). Therefore we concluded that cortical networks can be highly active during unconsciousness, but this activity is concentrated in short periods that are followed by profound suppression.

### Unconscious State is Marked by a Rapid Increase and Stable Maintenance of Power in the Slow Oscillation Band

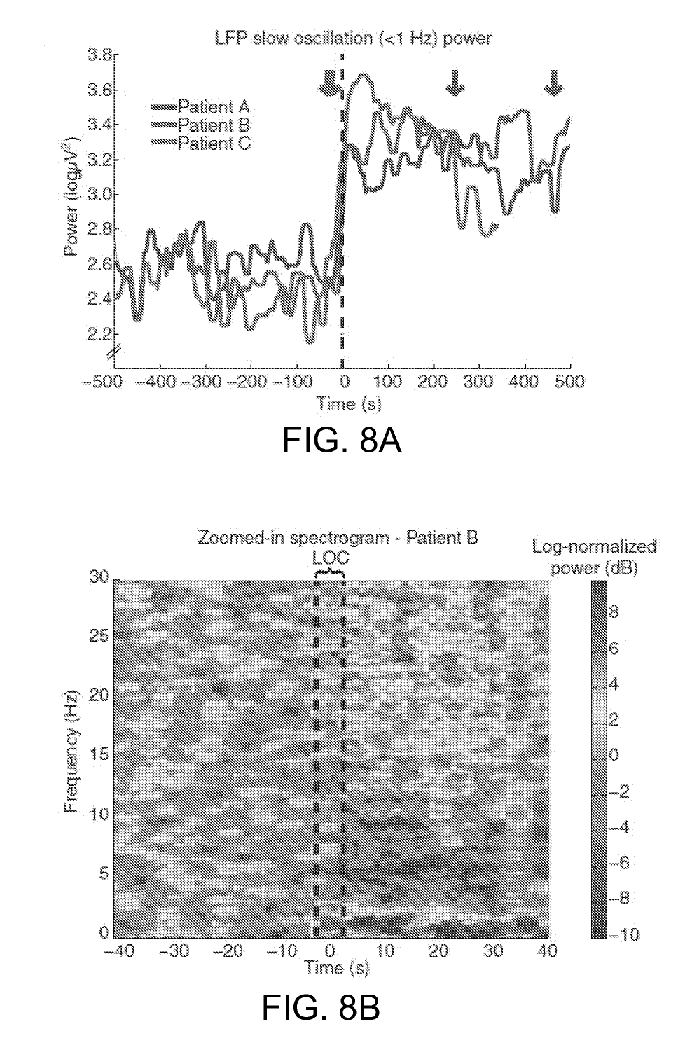

The slow oscillation is known to modulate neuronal spiking, and therefore we examined the time course of its onset relative to LOC. Before LOC, power in the slow oscillation band (0.1-1 Hz) was stable (SD<7% in each patient before LOC). At LOC, power in the slow oscillation band increased abruptly by 35-70% (FIG. 8), and this power increase occurred within one 5-s window of LOC in all patients (Table S1). The slow oscillation power then persisted at this high level for the remainder of the recording, with 99.0% of the post-LOC time bins having higher slow oscillation power than occurred in any time bin during baseline (FIG. 8A). We therefore concluded that power in the slow oscillation band is modulated simultaneously with LOC and is preserved thereafter despite large fluctuations in spike rate.

We next examined other frequency bands to investigate whether the power change at LOC was specific to the slow oscillation band or whether other frequency bands showed a similar relationship. Although power in the >10 Hz range increased slowly after LOC, theta (3-8 Hz) power showed the opposite trend, decreasing 20-30% after LOC (FIG. 8B). In addition, power in all these bands continued to undergo modulations for several minutes rather than maintaining a consistent change after LOC, perhaps as the result of differences in propofol dosage during the maintenance phase. The stable increase in power at LOC therefore was specific to the slow oscillation band. These results demonstrated that both spike rates and many oscillatory features (gamma, alpha, theta) are highly variable after LOC. In contrast, slow oscillation power increased abruptly at LOC and remained elevated throughout the rest of the recording (FIG. 8A). Therefore we concluded that onset of power in the slow oscillation band is associated with the transition into unconsciousness, whereas other oscillatory features do not reach a steady state until minutes later and may reflect dynamic neural shifts at varying concentrations of propofol.

### Neuronal Spiking Becomes Phase-Coupled to the Slow Oscillation at LOC

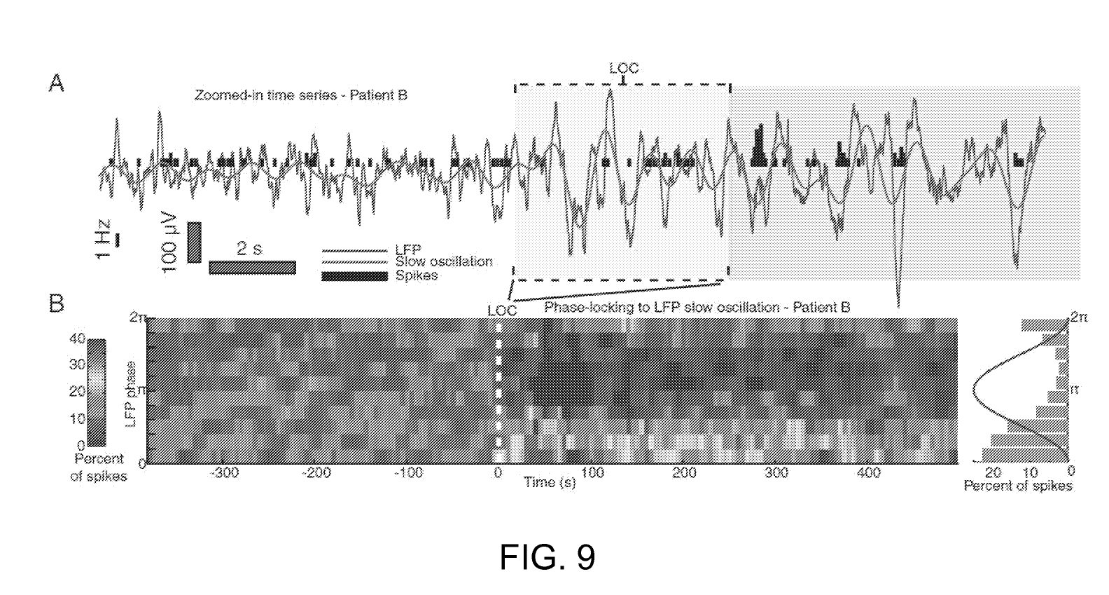

Studies of deeply anesthetized animals have shown that neuronal spike activity is coupled to the phase of the slow oscillation. We examined whether this spike-phase relationship developed immediately at LOC and whether it was maintained consistently thereafter. In each patient, population spike activity after LOC was significantly phase-coupled to the LFP slow oscillation (0.1-1 Hz), with 46.6% of spikes from all units occurring near the trough of the slow oscillation, during a phase of 0 to π/2 (maximum spiking at a phase of π/20-4π/20). Phase-coupling developed within seconds of LOC (between −2.5 and 7.5 s) and persisted throughout the ensuing changes in spike rate (FIG. 9). Spikes also were phase-coupled to the slow oscillation in the nearest ECoG channel but at a significantly different phase (maximum phase=0 to π/10; P<0.001, Kolmogorov-Smirnov test), suggesting that the LFP slow oscillation has a different relationship to spiking than the nearby, larger-scale ECoG recording. These results support the hypothesis that spikes become phase-coupled to the slow oscillation at LOC.

When examining individual units, most (67.2% of the 183 units with post-LOC spiking) were significantly phase-coupled to the LFP slow oscillation (P<0.05, Pearson's X² test). When this analysis was restricted to units with post-LOC spike rates over 0.1 Hz, 4.0% of units had significant phase coupling (P<0.05, n=50, Pearson's X² test). Of the units without significant phase-coupling, 65.0% also showed peak spiking activity within a phase of 0 to π/2, demonstrating that most units had the same phase-coupling trend. These results demonstrated that after LOC nearly all spiking activity is tightly coupled to the slow oscillation phase and is suppressed for a large portion of the slow oscillation cycle. We refer to these periods of high spiking as “ON” states and the silent periods as “OFF” states to remain consistent with previous work using only extracellular recordings. Because of the alternation of ON and OFF states, spike activity was limited to periods of a few hundred milliseconds, interrupted by periods of silence that also can last hundreds of milliseconds. Therefore we concluded that the slow oscillation marks a temporal fragmentation of cortical spiking that occurs at LOC.

### Slow Oscillation Impairs Information Transfer Between Distant Cortical Regions

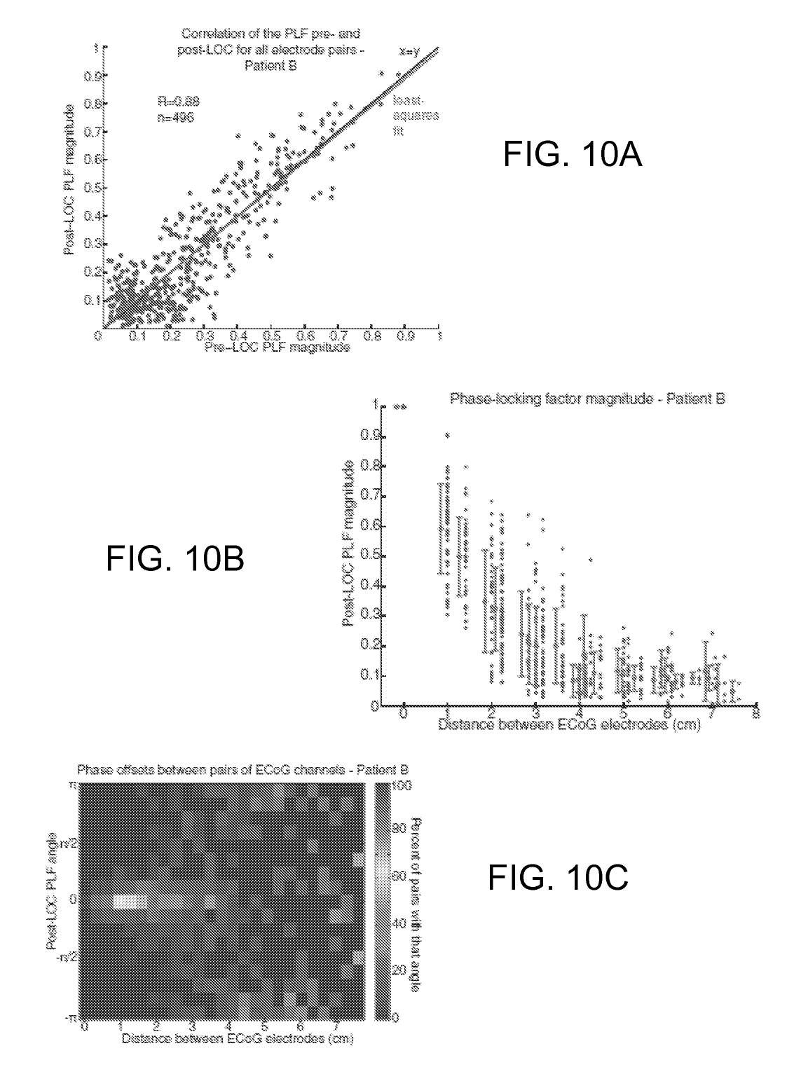

Given that post-LOC spiking is interrupted periodically within a cortical region, we investigated whether communication across distant areas also was affected. We examined slow dynamics across the grid of ECoG electrodes in the two patients (A and B) for whom we had at least 3 min of post-LOC ECoG data. FIG. 10A illustrates the position of ECoG and microelectrorecordings in patient B. Because spiking was strongly coupled to slow oscillation phase, we examined how this phase varied across the brain to infer the relative timing of neuronal activity in different cortical regions. We quantified the phase relationships between different cortical regions using the phase-locking factor (PLF), which characterizes the phase offset between two oscillations over a period. The PLF magnitude ranges between 0 and 1 and quantifies the stability of the phase offset (1 reflects constant phase offset; 0 represents variable phase offset). The PLF angle indicates the average phase offset. We calculated the PLF between every pair of ECoG channels on the grid (8×4 or 8×8 cm, n=96 total electrodes) to determine the relationship between local and distant slow oscillations. We found that the PLF magnitude was conserved between the pre- and post-LOC states (correlation coefficient R=0.66, patient A; R=0.88, patient B; P<10-50 for each, t test), with a small but significant increase in PLF magnitude after LOC (mean increase=0.02-0.07, P<0.01, Wilcoxon signed rank test) (FIG. 10A). This result was consistent with previous findings that low-frequency correlations in neural activity are maintained after LOC and suggests that LOC is associated with only a slight shift in the strength of phase relationships between slow oscillations in different areas.

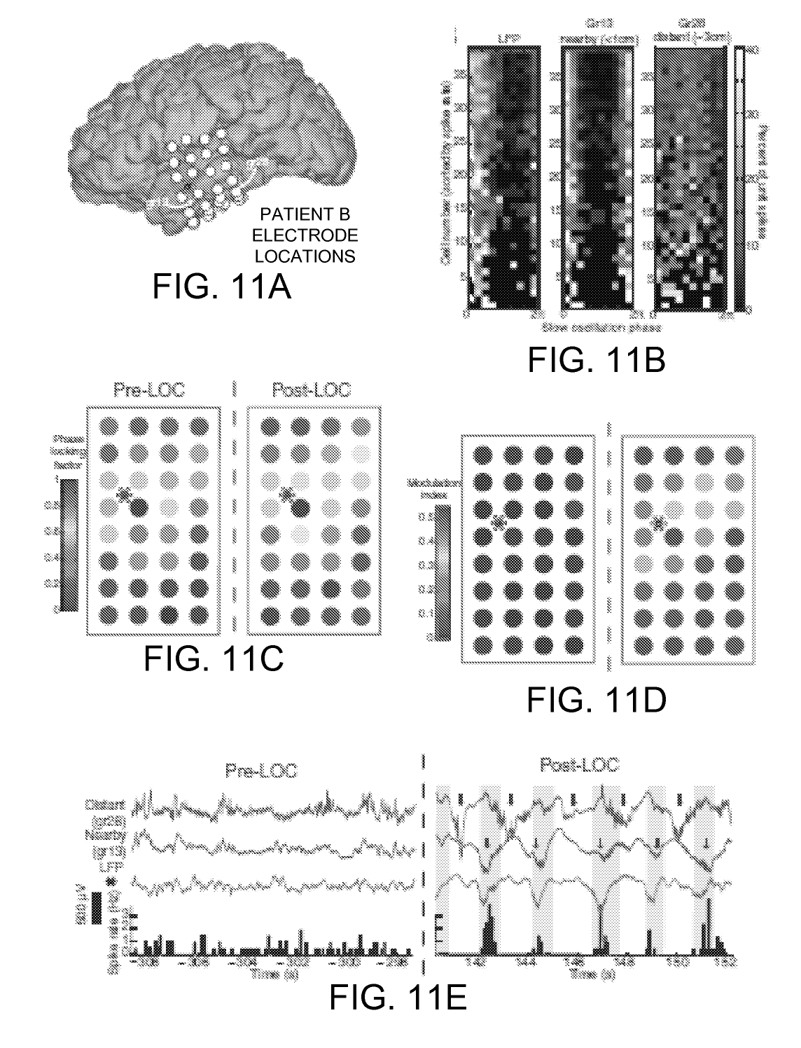

We next examined how the PLF varied with distance to determine whether slow oscillations in different cortical regions were at different phases. The PLF magnitude dropped significantly with distance (R=−0.61, patient A; R=−0.82, patient B; P<10⁻⁶ for each) (FIG. 10B and FIG. 11C), demonstrating that the phase offsets between distant slow oscillations were variable. We also examined the mean phase offsets (PLF angle). Mean phase offsets between distant channels varied across a wide range, spanning 0 to π (FIG. 10C). Because a phase offset of just π/4 corresponds to a lag of ˜250 ms, slow oscillations in distant ECoG channels had substantial timing differences. These results demonstrated that distant slow oscillations often were at different phases than the local oscillation, and these phase differences were not stable across time.

To examine how these phase offsets would affect neuronal activity, we examined the phase relationship between local spiking and slow oscillations measured across the ECoG grid. We measured spike phase-coupling as a modulation index (MI) quantifying the Kullback-Liebler divergence, in bits, between the observed phase distribution and a uniform distribution. A large MI indicates a strong relationship between local spiking and ECoG phase, whereas an MI of zero indicates no relationship. In the pre-LOC period, MI values were consistently small across all ECoG channels (MI range: 0.001-0.04 bits) (FIG. 11D), demonstrating that slow oscillation phase was not associated with strong suppression of spiking in the pre-LOC period. After LOC, the MI was significantly more variable across channels (range: 0.006-0.62 bits, P<0.01 in each patient, Levene's test). Spikes were strongly phase-coupled to the slow oscillation in the nearest ECoG channel, and this relationship declined significantly with distance (R=−0.40, patient A; R=−0.68, Patient B; P<0.001 in each patient) (FIGS. 11B and 11D).

Taken together, our analysis of phase-phase and spike-phase coupling show that the post-LOC state is characterized by periodic and profound suppression of spiking coupled to the local slow oscillation phase and that this phase is not consistent across cortex. Given the strong relationship between phase and ON/OFF periods, this result suggests that, after LOC, ON periods in distant (>2 cm) cortical regions occur at different times (FIG. 11E, Right). In contrast, low-frequency oscillations in the pre-LOC state are not associated with strong suppression of spiking, so neurons are able to fire at any phase of local or distant slow oscillations despite the presence of phase offsets (FIG. 11E, Left). The combination of phase offsets and strong phase-coupling of spikes that occurs at LOC therefore is expected to disrupt communication between distant cortical areas, because one cortical area frequently will be profoundly suppressed when another area is active.

Although spikes were not strongly phase-coupled to distant slow oscillations during the post-LOC period, several electrodes located more than 3 cm from the spike recording site showed a statistically significant relationship. In these cases, phase-coupling was weak, and the phase of maximal spiking was shifted, consistent with our conclusion that distant cortical regions are unlikely to have simultaneous ON periods. However, this finding raises the possibility that, despite the asynchrony of slow oscillations across the brain, there might still be a link between slow oscillations in distant cortical regions. Given the observed phase offsets (which ranged up to π), such coupling would occur frequently over hundreds of milliseconds and would not reflect precisely timed inputs and interactions. Overall, these analyses support the conclusion that distant cortical regions frequently were at a suppressed phase of the slow oscillation when the local network was active. Therefore activity within a cortical area was isolated, impairing communication between distant regions.

### Local Network Structure is Preserved After LOC

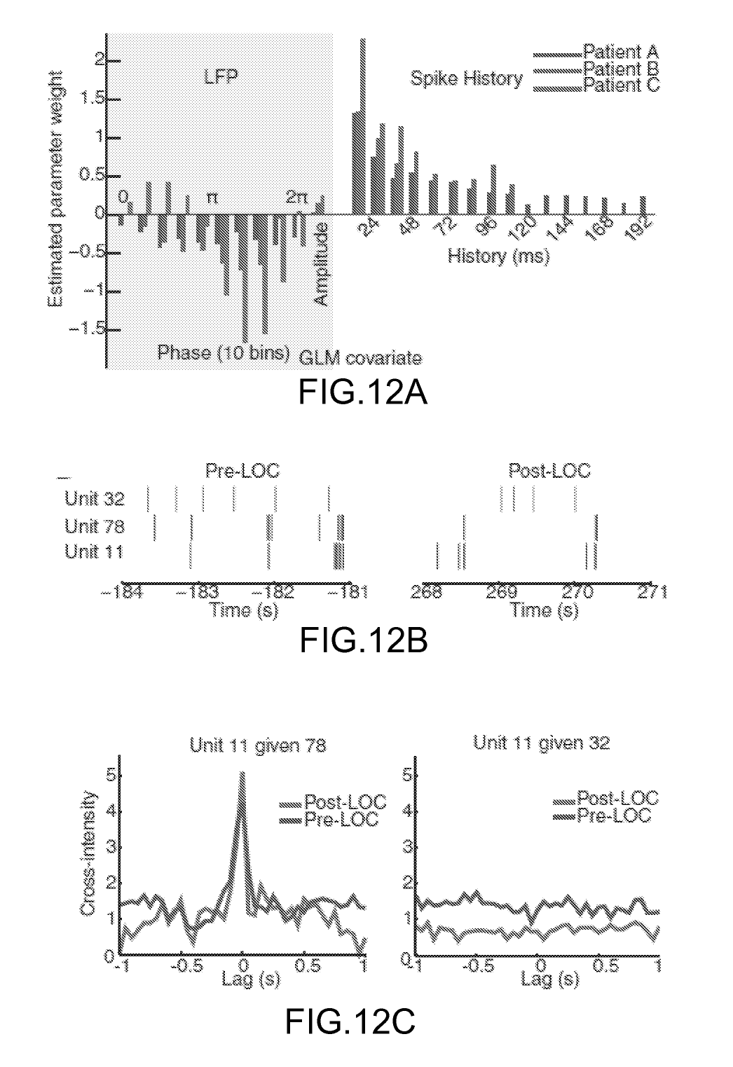

Having observed interruptions in local activity and disruption of long-range communication, we examined whether connectivity within the local cortical network also was impaired. We fit a generalized linear model (GLM) to spike activity from the ensemble of units to test whether spiking could be predicted by the slow oscillation phase alone or whether the history of local network activity also contributed. We used the Bayesian Information Criterion to select the number of covariates to include in the model. In each patient, we found that this model included >30 ms of population spike history (FIG. 12A). Ensemble spike history therefore predicted future spiking, demonstrating that, although cortical activity was limited to brief ON periods, inter-unit structure existed within these periods. This pattern resembled the pre-LOC state, in which recent spike history (0-48 ms) was predictive of future spikes and more distant spike history contributed less. This result suggests that, after LOC, cortical activity is not reduced to disordered spiking during ON periods. Instead, significant structure is maintained between nearby neurons during their brief periods of activity.

Structure between single units was reflected further in a peak in the cross-intensity function between several pairs of units, demonstrating millisecond-scale synchronization of spike activity (FIGS. 12B and 12C). To examine whether pair-wise synchronization persisted after LOC, we analyzed the cross-correlation between the 15 units with the highest spike rates in patient A. Of the 103 pairs (excluding pairs recorded on the same electrode), 21 were significantly correlated before LOC (P<0.05, exact Poisson test relative to baseline from shuffled spikes, Bonferroni correction for multiple comparisons). After LOC, 71.4% of these pairs remained significantly correlated. In contrast, a significantly smaller number (only 18.3%) of pairs that were not correlated before LOC became correlated after LOC (P<10−5, Fisher's exact test). This result demonstrated that pairs of units tended to retain the same correlation structure after LOC that they had before LOC, whether it was the presence or absence of a correlation. Taken together, both the GLM and paired correlation results show that significant inter-unit connectivity is maintained within post-LOC ON periods. This result suggests that the dominant change after LOC is the isolation of cortical networks, whereas aspects of local network structure may remain unaltered.

Spiking Activity is Associated with Modulations in Slow Oscillation Shape and Higher Frequency Power

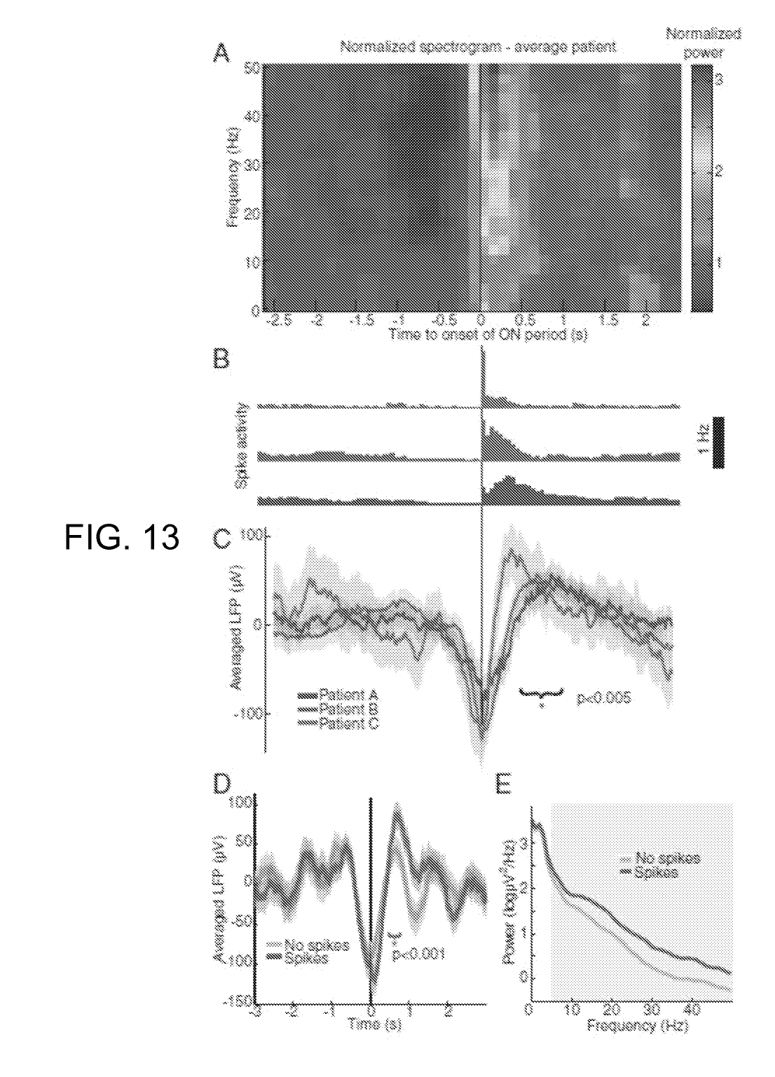

The mechanisms underlying the slow oscillation are debated. Therefore, we examined the relationship between spike activity and slow oscillation shape in greater detail. FIG. 13A shows a normalized spectrogram for the average patient. We calculated an average LFP triggered at the beginning of ON periods. The triggered average demonstrated that ON periods begin at the minimum of the LFP slow oscillation (FIGS. 13B and 13C). In addition, the LFP slow oscillation was asymmetric (FIG. 13C), with a higher peak after spiking than before spiking (mean difference=40.7 μV, P<10−5, P<0.005 for each patient, t-test). We tested whether this asymmetric shape occurred on all cycles of the slow oscillation or was specific to cycles with high spike activity. We compared cycles of the LFP slow oscillation that contained spikes with cycles that did not, matching the amplitudes of the slow oscillation minimum. Cycles that were not associated with spikes were symmetric (mean difference=0.3 μV, P>0.9, t test), whereas those that were associated with many spikes produced a higher peak after spiking (mean difference=32.2 μV, P<0.001, P<0.05 for each patient, t-test) (FIG. 13D). This asymmetry did not extend to the nearby ECoG recording, suggesting that the relationship between spike activity and slow oscillation shape is a highly local effect limited to less than 1 cm (i.e., the spacing in the ECoG grid). These results demonstrated that high spike rates are associated with an increased slow oscillation peak in the LFP, potentially reflecting enhanced suppression after spike activity.

Because low gamma (25-50 Hz) power also increased after LOC, we examined its relationship to spike activity as well. ON periods were associated with significantly increased broadband (<50 Hz) power in the LFP and ECoG (P<0.05, F-test, Bonferroni correction for multiple comparisons across frequencies) (FIG. 13A). LFP power in alpha, beta, and gamma bands was significantly higher in slow oscillation cycles with high spike activity than in cycles with low spike activity (P<0.05, F-test, Bonferroni correction for multiple comparisons across frequencies) (FIG. 13E). These results showed that, in addition to the slow changes in gamma power occurring over minutes, gamma power also fluctuated at the timescale of the slow oscillation (0.1-1 Hz) and was higher during ON periods. Therefore we concluded that after LOC power in the low gamma range is associated with high local spike rates. This result suggested that the gradual increase in gamma power after LOC may be related to the post-LOC fluctuations in spike rate rather than reflecting dynamics induced specifically at LOC.

Slow oscillations during propofol-induced unconsciousness share several features with slow waves during sleep, namely, in both states, spike activity is coupled to a local slow oscillation that is not synchronous across the brain. The asynchronicity observed herein contrasts with previous observations in anesthetized animals and is most likely caused by the increased spatial sampling provided by the 8-cm grid of intracranial electrodes. In addition, the preservation of pre-LOC neuronal network properties after LOC is consistent with the hypothesis that cortical UP states during sleep have dynamics similar to the waking state. However, the patterns observed under propofol also show striking differences from patterns during sleep.

The abrupt onset of the slow oscillation during propofol induction of general anesthesia, induces rapid LOC caused by a bolus administration. Since general anesthesia is typically induced with a bolus, the abrupt transition into the slow oscillation is likely to occur in the majority of clinical patients when losing consciousness during general anesthesia. By contrast, during sleep, the slow oscillation develops over minutes, consistent with the gradual nature of the transition into sleep. In both cases, slow oscillation dynamics temporally track LOC, further supporting the proposal that the slow oscillation represents a breakdown of cortical communication. In addition, periods of spike activity were brief in present results, whereas sleep is characterized by persistent spiking with brief periods of suppression during slow-wave events. A difference in the ratio of UP and DOWN states could provide one explanation for why propofol creates a more profound disruption of consciousness than sleep, namely a possible reduced temporal overlap in neuronal spiking between different cortical regions, more reliably preventing the organization of large-scale population activity. Furthermore, recent findings that isolated OFF states in sleep-deprived rodents are associated with behavioral impairment are consistent with the hypothesis that the spatial and temporal properties of OFF states affect cortical function.

The relationships identified herein between spike activity and slow oscillation shape suggest that cortical spiking may have a causal role in the slow oscillation. Spikes predict a high-amplitude peak in the LFP slow oscillation, but this effect does not extend to the ECoG recordings, which integrate activity from a larger population of neurons. The highly local nature of this effect suggests that cortical spiking may affect the local slow oscillation directly. One possible mechanism is that pyramidal neuron spiking during ON periods excites GABAergic inter-neurons, whose inhibitory actions are enhanced by propofol, driving the local network into a more hyperpolarized state. Another possibility is that spike activity may drive disfacilitation of cortical neurons, a mechanism that has been demonstrated in slow-wave sleep. These effects could be consistent with either the cortical or corticothalamic hypothesis.

Moreover, slow oscillation dynamics may also relate to observed gamma coherence decreases after propofol-induced LOC, particularly across distant cortical regions, since spiking activity was shown to be strongly associated with gamma power, and spiking is unlikely to occur simultaneously in distant cortical regions because of the asynchronicity of slow oscillations across the brain. Slow oscillations may therefore impair coupling of gamma oscillations between cortical areas, and this effect could produce gamma oscillations that are not coherent over long distances. Low-frequency spatial correlations in fMRI and ECoG, sometimes used to assess functional connectivity, have been found to remain invariant after LOC under propofol. Analysis of the PLF magnitude, in accordance with the present disclosure, has a similar spatial distribution before and after LOC, corroborating previous observations. Studies shown herein demonstrate that although the low-frequency spatial relationships remain similar before and after LOC, the functional properties of low-frequency oscillations change at LOC, grouping spiking into brief ON states that are disjoint across space.

It is also noteworthy that patients enrolled in this study had epilepsy, and it is possible that their cortical networks differed because of seizure foci or medication history. However, several factors support the hypothesis that these results generalize to the healthy brain. First, the microelectrodes were located at least 2 cm from the seizure focus in each patient, and histology did not reveal any disruption of the local network, suggesting that the LFPs and single units were recorded from healthy cortex. Second, the overall effects of propofol were highly consistent with those observed in healthy subjects, namely, that unconsciousness was associated with increased slow oscillation power and increased gamma power, in strong agreement with previous studies. These results suggest that propofol acted typically in these patients' brains. Finally, we report statistics for each individual patient and show that the timing of the slow oscillation onset and its relationship to spiking were replicated across patients despite their individual clinical profiles. Because epilepsy is a heterogeneous disease with different cortical origins, the high consistency of these results suggests that the effects reported here are not caused by the presence of epilepsy. These three observations suggest that our results are not a product of an epileptic brain but rather reflect a true neural correlate of LOC that is likely to generalize to the healthy brain.

### Example II

Although some EEG patterns are observed consistently during certain procedures, it is unclear how they are functionally related to unconsciousness. Specifically, other anesthetic drugs, such as ketamine and dexmedetomidine, operate through molecular and neural circuit mechanisms that may be different from those of propofol. For example, similar EEG patterns are known to arise for different drugs, such as with propofol, an γ-Aminobutyric acid receptor-specific agonist (GABA_{A}), and dexmedetomidin, an α2-adrenoceptor agonist. Propofol is associated with well-coordinated frontal thalamocortical alpha oscillations and asynchronous slow oscillations. Similarly, dexmedetomidine gives rise to spindle-like activity detected in the 8-12 Hz range over the frontal region and slow oscillations. As such, although EEG patterns observed during administration appear superficially similar, different behavioral or clinical properties may be exhibited. For example, unlike patients receiving propofol, patients receiving an infusion of dexmedetomidine can be easily aroused with gentle verbal or tactile stimuli at blood concentration levels required to maintain loss of consciousness (LOC). This leads to the natural question of whether there are differences in the brain dynamics induced by different drugs that can explain the observed differences in clinical response and behavior, and whether such brain dynamics can be detected in the EEG.

To investigate shared relationships between the EEG activity of dexmedetomidine and propofol, and altered states of arousal, intraoperative frontal EEG were recorded from patients undergoing light sedation with dexmedetomidine, sedation with propofol and general anesthesia (GA) with propofol. As described below, EEG dynamics, using time-varying spectral, and coherence methods revealed that, although the mean group level spectrograms appeared qualitatively similar, the patterns of coherence in the 0.1-1 Hz and 8-12 Hz EEG frequency bands were different. Dexmedetomidine induces 0.1-1 Hz slow oscillations that exhibited greater coherence compared to propofol slow oscillations. This finding is consistent with the observation that sleep-related slow oscillations are highly synchronous, while propofol-induced slow oscillations are asynchronous and reflect a state of fragmented cortical communication. Conversely, dexmedetomidine induced 8-12 Hz oscillations exhibited less coherence than propofol induced oscillations. This is consistent with the notion that coherence of 8-12 Hz oscillations represents an entrainment of frontal thalamocortical circuits that block communication. Notably, these differences in coherence vary appropriately with the levels of consciousness represented by the three groups studied.

In addition, to study the relationship between EEG dynamics in context of potential neural circuit mechanisms of an anesthetic vapor, intra-operative EEG were recorded from patients undergoing general anesthesia with sevoflurane as the primary maintenance agent, which is an ether derivative commonly used to maintain GA. Unlike the intravenous anesthetic agent propofol, the EEG signature of sevoflurane, has not been well studied. Connectivity analysis was then performed on the EEG dynamics postulated to be involved in anesthesia-induce depression in consciousness. As described below, it was found that during GA induced unconsciousness the macroscopic EEG dynamics of sevoflurane closely resemble those of propofol. These observed similarities are consistent with present understanding of how EEG features relate to anesthesia-induced depression of consciousness, and provide a framework for further experimental studies on the neural circuit mechanisms of general anesthesia.

### Methods

### Spectral Analysis

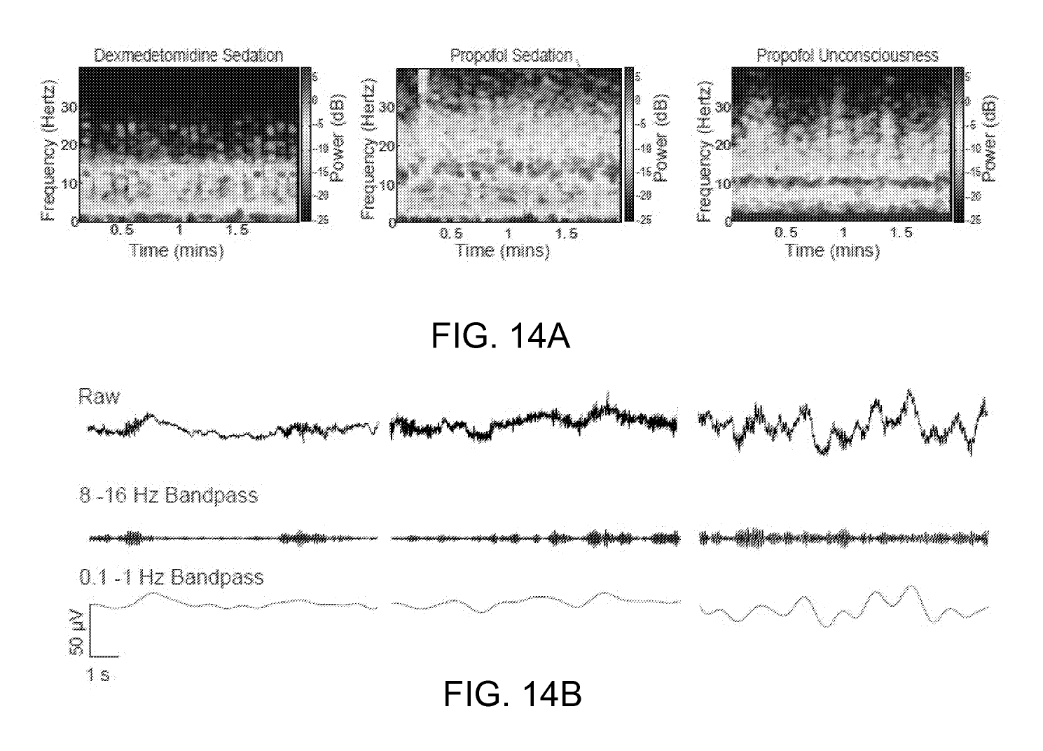

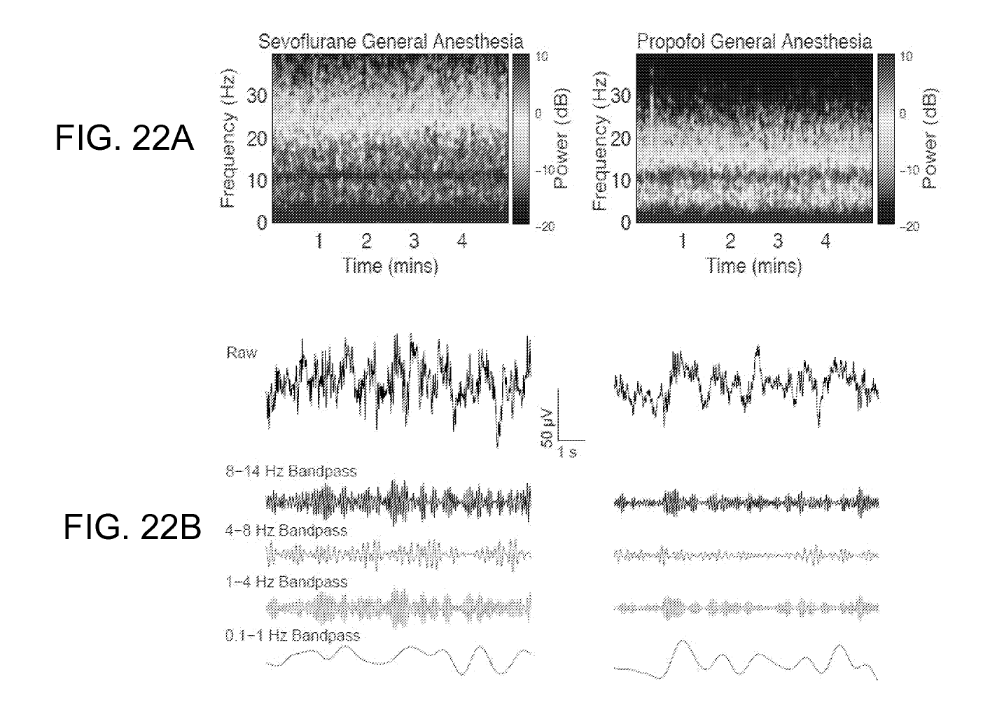

The power spectral density, also referred to as the power spectrum or spectrum, quantifies the frequency distribution of energy or power within a signal. The spectrogram is a time-varying version of the spectrum. For example, FIG. 14A and FIG. 22A show representative volunteer EEG spectrograms under dexmedetomidine sedation, propofol sedation and propofol-induced unconsciousness, and sevoflurane-induced general anesthesia. In these spectrograms, frequencies are arranged along the y-axis, and time along the x-axis, and power is indicated by color on a decibel (dB) scale. FIG. 14B and FIG. 22B selected epochs of 0.1-1 Hz, 1-4 Hz, 4-8 Hz and 8-16 Hz bandpass filtered EEG signals in the time domain. Spectra and spectrograms were computed using the multitaper method, implemented in the Chronux toolbox (http://chronux.orq). The multitaper method was chosen specifically because it allows the spectral resolution to be set precisely, which is desirable in observing many anesthesia-related phenomena. Moreover, for a particular choice of spectral resolution, the multitaper method offers lower bias and lower variance than traditional nonparametric spectral estimation methods. Such lower bias and variance results in displays that are visually clearer, with oscillations or peaks that are more distinct, and facilitates greater sensitivity and specificity in subsequent processing or inference steps.

In general, anesthesia-related oscillations have a bandwidth of approximately 0.5 to 1 Hz for slow and alpha and spindle oscillations. Anesthesia-induced beta and gamma oscillations tend to be wider, approximately 5 Hz or more in bandwidth. The spectral analysis parameters can be chosen to make these oscillations clearly visible and distinguishable from one another, while also ensuring sufficient temporal resolution to track time-varying changes. For instance, if narrower spectral resolution were required, a longer window length T could be chosen, but with the tradeoff that rapid time-varying changes would be more difficult to discern. Similarly, the time-bandwidth product TW could be reduced to improve spectral resolution, but with the tradeoff that fewer tapers could be used (K<=2TW−1), resulting in increased variance. Similarly, a shorter window length T could be chosen to improve temporal tracking, and a wider time-bandwidth product TW could be chosen to improve variance, both with the tradeoff of lower spectral resolution. In general, these spectral analysis parameters can be varied from the example provided here in order to enhance or optimize detection, visualization, and temporal tracking of the anesthetic or sedative properties of interest. Moreover, different sets of parameters could be used or made available for different drugs or clinical scenarios.

Group-level spectrograms were computed by taking the median across volunteers. Spectra were also calculated for selected EEG epochs. The resulting spectra were then averaged for all epochs, and 95% confidence intervals were computed via taper-based jackknife techniques. The spectral analysis parameters included window length T=4 s with 0 s overlap, time-bandwidth product TW=3, number of tapers K=5, and spectral resolution 2 W of 1.5 Hz. Peak power, and its frequency, was estimated for the dex-spindle, travelling peak, and frontal alpha oscillations for each individual subject. Averages across subjects were performed to obtain the group-level peak power and frequency for these oscillations.

### Coherence Analysis

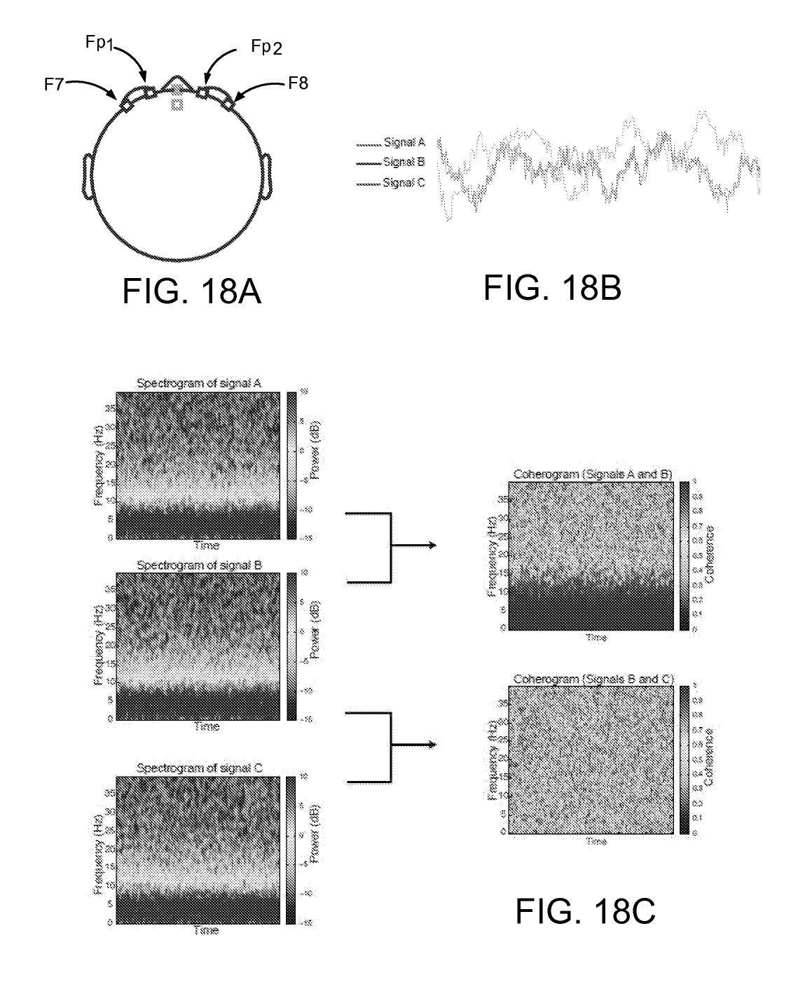

The coherence quantifies the degree of correlation between two signals at a given frequency. It is equivalent to a correlation coefficient indexed by frequency: a coherence of 1 indicates that two signals are perfectly correlated at that frequency, while a coherence of 0 indicates that the two signals are uncorrelated at that frequency. The coherence C_{xy}(f) function between two signals x and y is defined as:

$${C}_{xy} ( f ) = \frac{\vert {S}_{xy} ( f ) \vert}{\sqrt{{S}_{xx} ( f ) {S}_{yy} ( f )}}$$

where S_{xy}(f) is the cross-spectrum between the signals x(t) and y(t), Sₓₓ(f) is the power spectrum of the signal x(t) and S_{yy}(f) is the power spectrum of the signal y(t). Similar to the spectrum and spectrogram, the coherence can be estimated as a time-varying quantity called the coherogram. Coherograms were computed between two frontal EEG electrodes, namely F7 and F8 (FIG. 18A), using the multitaper method, implemented in the Chronux toolbox (http://chronux.org). The multitaper method was chosen specifically because it allows the spectral resolution to be set precisely, which is required to observe many anesthesia-related phenomena. Moreover, for a particular choice of spectral resolution, the multitaper method offers lower bias and lower variance than traditional nonparametric spectral estimation methods. Such lower bias and variance results in displays that are visually clearer, with oscillations or peaks that are more distinct, and facilitates greater sensitivity and specificity in subsequent processing or inference steps. Group-level coherograms were computed by taking the median across volunteers. Coherence was also calculated for the selected EEG epochs. The resulting coherence estimates were then averaged for all epochs, and 95% confidence intervals were computed via taper-based jackknife techniques. The coherence analysis parameters were: window length T=4 s with 0 s overlap, time-bandwidth product TW=3, number of tapers K=5, and spectral resolution 2 W of 1.5 Hz. Peak coherence, and its frequency, was estimated for the dex-spindle, travelling peak, and frontal alpha oscillation for each individual subject. Averages across subjects were performed to obtain the group-level peak coherence and frequency for these oscillations. The coherence provides information equivalent to the magnitude of the PLF, as described. Thus, the changes in low-frequency coherence described below reflect the same changes in cortical dynamics described above in terms of the PLF.

### Statistical Analysis

To compare spectral and coherence estimates between groups, jackknife-based methods were used, namely the two-group test for spectra (TGTS), and the two-group test for coherence (TGTC), as implemented by the Chronux toolbox (http://www.chronukorg). This method accounts for the underlying spectral resolution of the spectral and coherence estimates, and considers differences to be significant if they are present for contiguous frequencies over a range greater than the spectral resolution 2 W. Specifically, for frequencies f>2 W, the null hypothesis was rejected only if the test statistic exceeded the significance threshold over a contiguous frequency range ≧2 W. For frequencies 0≦f≦2 W, to account for the properties of multitaper spectral estimates at frequencies close to zero, the null hypothesis was rejected only if the test statistic exceeded the significance threshold over a contiguous frequency range from 0 to max(f,W)≦2 W. A significance threshold of p<0.05 was selected for within group comparisons and p<0.001 for between group comparisons, applying a Bonferonni correction for multiple comparisons where appropriate.

### Results

### Propofol and Dexmedetomine-Induced EEG Patterns

During induction and emergence from dexmedetomidine sedation, we recorded EEGs using a 64-channel BrainVision MRI Plus system (Brain Products) with a sampling rate of 1,000 Hz, resolution 0.5 pV least significant bit (LSB), bandwidth 0.016-1000 Hz. Volunteers were instructed to close their eyes throughout the study to avoid eye-blink artifacts in the EEG. Volunteers were presented with auditory stimuli during the study and asked to respond by button presses to assess the level of conscious behavior. The stimuli consisted of the volunteer's name presented every two minutes. Button-press stimuli were recorded using a custom-built computer mouse with straps fitted to hold the first and second fingers in place over the mouse buttons. The mouse was also lightly strapped to the subject's hand using tape and an arterial line board to ensure that responses could be recorded accurately.

We applied an anti-aliasing filter and down-sampled the EEG data to 250 Hz before analysis. EEG signals were re-montaged to a nearest-neighbor Laplacian reference, using distances along the scalp surface to weigh neighboring electrode contributions. First, 2-minute EEG segments were selected from all subjects during the awake, eyes closed baseline. Eye closure facilitates distinguishing between normal awake, eyes-closed occipital alpha oscillations and the frontal alpha oscillations associated with anesthesia induced altered arousal. EEG data segments were selected based on the behavioral response.

For dexmedetomidine, the onset of sedation was defined as the first failed behavioral response that was followed by a series of at least three successive failures. To characterize the EEG signature of dexmedetomidine sedation, we used the first 2-minute EEG epoch obtained for each volunteer 6-minutes after the onset of sustained sedation.

For propofol, we identified data segments using a combination of behavioral and electrophysiological endpoints. In previous work, we discovered two forms of propofol-induced phase-amplitude modulation, referred to as trough-max and peak-max. In the trough-max pattern, propofol-induced alpha waves are strongest at the troughs of the slow oscillation. This pattern arises during the transitions to and from unconsciousness, and bisects unconsciousness defined by loss of response to auditory stimuli. As such, clinically, onset of the trough max pattern marks the earliest part of the continuum of propofol sedation that we could identify. For each volunteer subject, we chose trough max EEG epochs that occurred within the first 2 minutes of the onset of this pattern. In the peak-max pattern, propofol-induced alpha waves are strongest at the peaks of the slow oscillation. This pattern arises after loss of consciousness, when the probability of response to auditory stimuli is zero. It signifies a profound state of unconsciousness. Hence, peak-max is clinically similar to unconsciousness during general anesthesia. From here, we refer to the trough-max state as “sedation,” and the peak-max state as “unconsciousness.”

Dexmedetomidine vs. Baseline Power Spectra

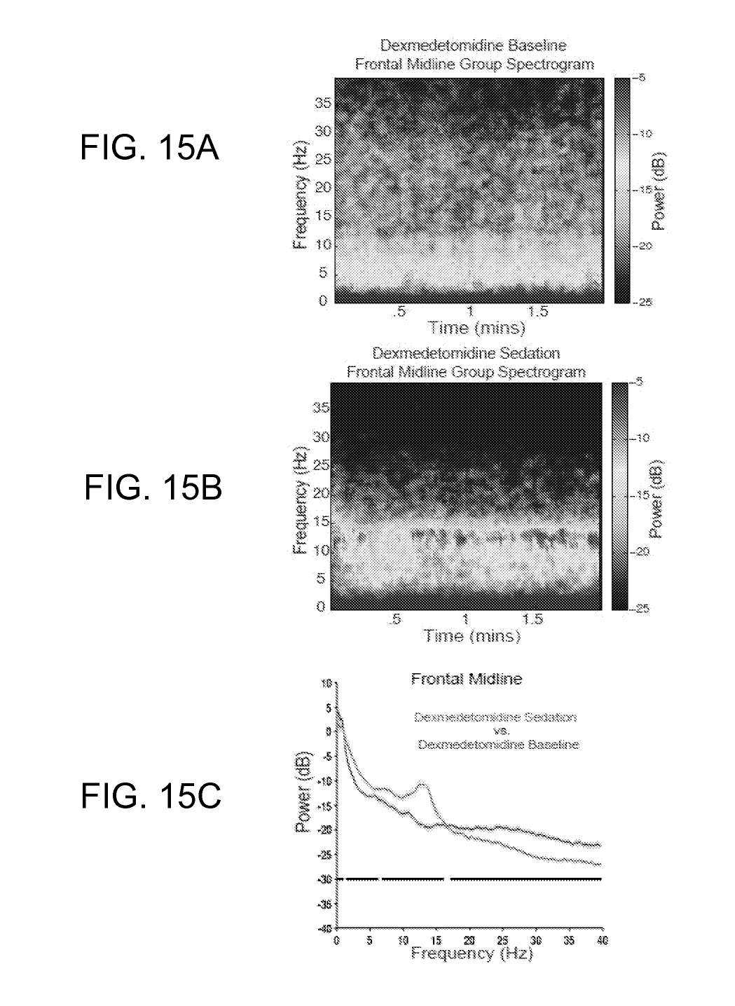

We observed differences in the spectrogram of dexmedetomidine sedation and dexmedetomidine baseline. In particular, the dexmedetomidine sedation spectrogram showed a robust visually evident increase in power across a frequency range of 2-15 Hz (FIG. 15A, 15B). We next compared the EEG spectrum during dexmedetomidine sedation and baseline, and found significant differences in power across most frequencies between 0 and 40 Hz. EEG power exhibited a dex-spindle oscillation peak (mean±std; peak frequency, 13.1 Hz±0.86; peak power, −10.2 dB±.3.2), and was higher during dexmedetomidine sedation across a range of frequencies less than 16.4 Hz (FIG. 15C; 0.1-1.2 Hz, 1.7-6.6 Hz, 7-16.4 Hz; P<0.05, TGTS). EEG power was also lower during dexmedetomidine sedation in beta/gamma frequency ranges (FIG. 15C; 17.4-40 Hz; P<0.05, TGTS). Our results show that, compared to the awake-state, spindle-like oscillations (dex-spindles) are exhibited during dexmedetomidine sedation.

Propofol vs. Baseline Power Spectra

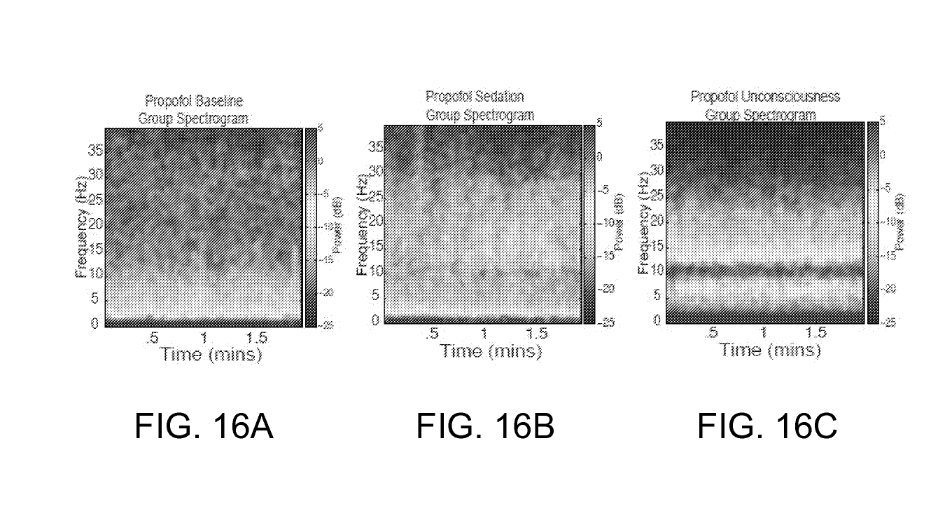

Compared to baseline, we also observed differences in the spectrogram during propofol sedation and propofol-induced unconsciousness. Propofol sedation was characterized by broad-band (˜1-25 Hz) increased power whereas during propofol-induced unconsciousness, the increased power appeared confined to slow, delta and alpha frequency bands (FIG. 16A, 16B, 16C). Qualitatively, during propofol-induced unconsciousness, the EEG spectrogram exhibited a visibly narrower 8-12 Hz oscillation bandwidth compared to propofol sedation (FIGS. 16B, 16C). Our results are consistent with previous reports that frontal alpha oscillations are exhibited during propofol-induced unconsciousness, and that higher-frequency beta-gamma oscillations are observed during propofol sedation.

Dexmedetomidine vs. Propofol Power Spectra

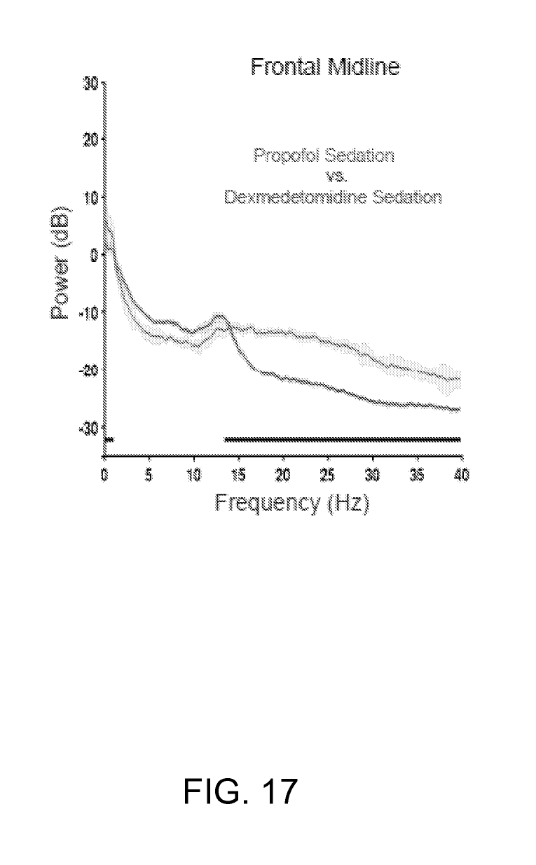

Next we compared the spectra during dexmedetomidine sedation, propofol sedation, and propofol-induced unconsciousness. We found that EEG power was greater during propofol sedation compared to dexmedetomidine sedation across a broad frequency range spanning slow, beta and gamma frequencies (FIG. 17; 0.1-1.2 Hz, 12.9-40 Hz; P<0.0005, TGTS). Qualitatively, the spectrum during dexmedetomidine sedation showed a clear dex-spindle peak at ˜13 Hz, while propofol sedation did not exhibit a clearly distinguishable peak. Slow oscillations during propofol-induced unconsciousness (power, 19.2 dB±2.4) were almost an order of magnitude larger than during dexmedetomidine sedation (power, 1.8 dB±1.6). Similarly, propofol-induced frontal alpha oscillations (power, 2.5 dB±3.8) were also larger than the dex spindles (power, −10.2 dB±3.2). Our results show that the spindle-like EEG pattern induced by dexmedetomidine is a dynamic pattern distinct from the propofol-induced travelling peak and frontal alpha oscillations. In addition, propofol-induced slow oscillations are much stronger than those produced by dexmedetomidine.

To illustrate how the coherogram quantifies relationships between signals, and how this is distinct from the spectrogram, we devised a simulated data example. FIG. 18A shows time domain traces from three simulated oscillatory signals, two of which are highly correlated (signal A and signal B), and one which is uncorrelated with the other two (signal C). FIG. 18C shows the spectrograms (left) and coherograms (right) for these signals. All three signals have identical spectrograms, by construction, but the coherence between the signals is very different, reflecting the presence or absence of the visible correlation evident in the time domain traces. The coherogram also indicates the frequencies over which two signals are correlated. In the example in FIG. 18B, signals A and B are correlated at frequencies below approximately 20 Hz. This example shows how the cohereogram characterizes the correlation between two signals as a function of frequency. The coherence can be interpreted similarly.

Dexmedetomidine vs. Baseline Coherence

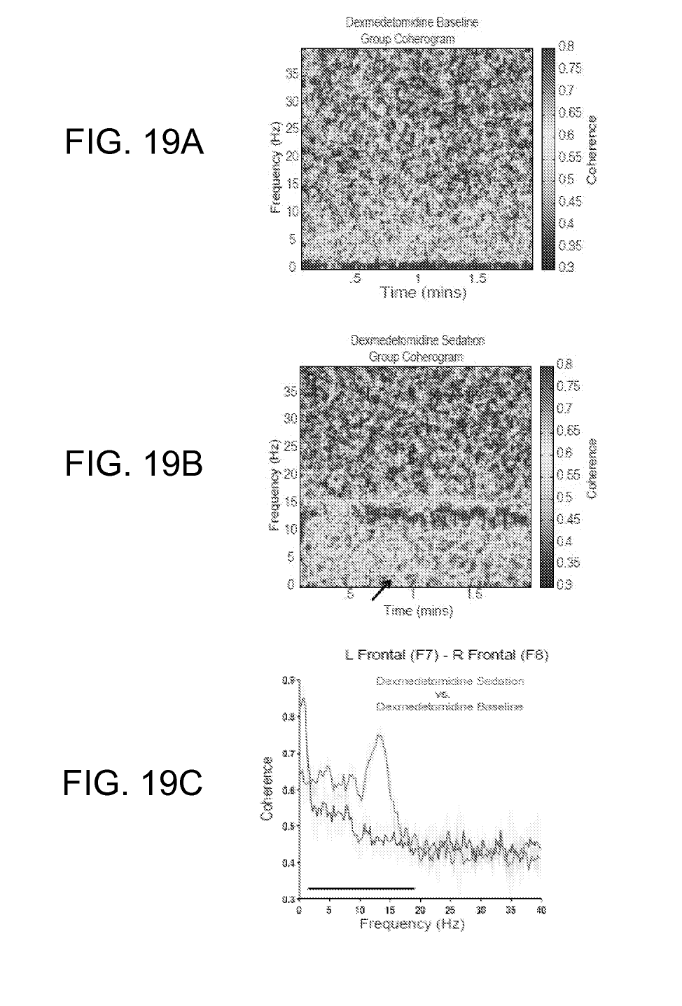

Compared to baseline, we observed differences in slow oscillation coherence in the coherogram during dexmedetomidine sedation. In particular, dexmedetomidine sedation was characterized by increase in coherence across a frequency range of 1-15 Hz (FIGS. 19A and 19B) and a decrease in 0.1-1 Hz coherence (solid arrow, FIG. 19B). We compared the EEG coherence during dexmedetomidine sedation and baseline, and found significant differences in coherence across frequencies between 0.1 and 19.3 Hz, with a coherence peak (peak frequency, 13.3 Hz±0.9; peak coherence, 0.78±0.08) consistent with the dex-spindle (FIG. 19C; 0.1-1.2 Hz; 1.7-19.3 Hz; P<0.05, TGTC). Our results show that compared to the awake-state, dexmedetomidine sedation was characterized by dex-spindles that were coherent and slow oscillations that were not coherent.

Propofol vs. Baseline Coherence

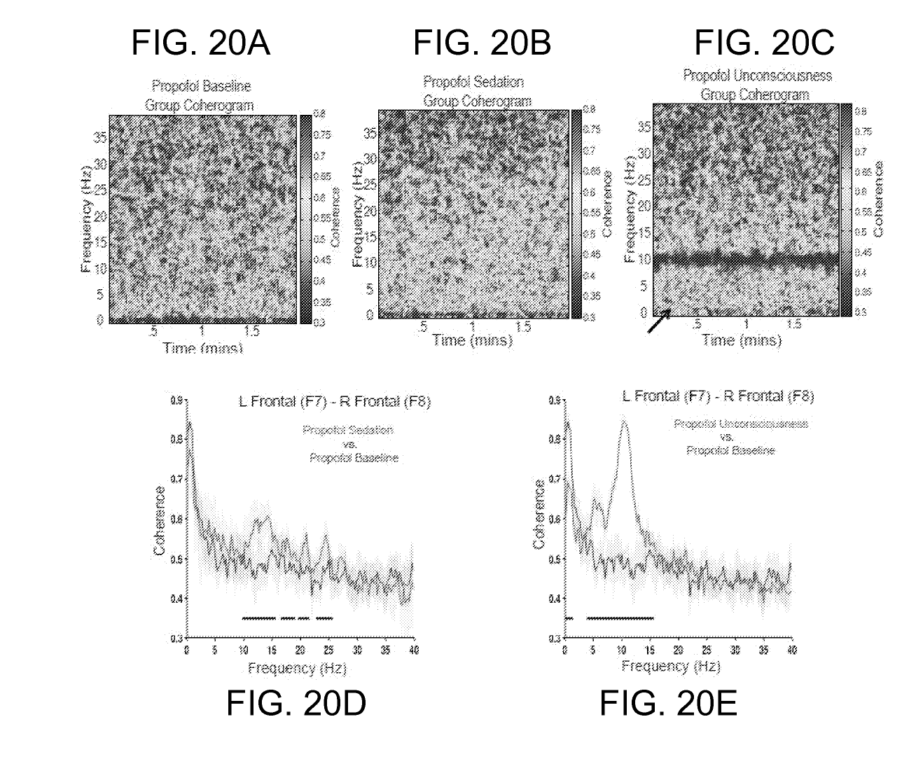

Compared to baseline, we also observed differences in the coherogram during propofol sedation and propofol-induced unconsciousness. Propofol sedation was characterized by a broad (˜1-25 Hz) increase in coherence on the coherogram. Propofol-induced unconsciousness was characterized by a narrow band of alpha oscillation coherence centered at ˜10 Hz (FIG. 20A, 20B, 20C) and a decrease in 0.1-1 Hz coherence (solid arrow, FIG. 20B). We next compared the coherence during sedation and unconsciousness to the baseline state. We found that there were discrete bands of coherent EEG activity (peak frequency, 16.1 Hz±4.9; peak coherence, 0.69±0.05) corresponding to the traveling-peak (FIG. 20D; 10-16 Hz, 16.8-19.3 Hz, 19.8-21.7 Hz, 22.9-25.9 Hz; P<0.025, TGTC). Notably, during propofol-induced unconsciousness, there was a distinct alpha oscillation coherence peak (peak frequency, 10.8.1 Hz±0.68; peak coherence, 0.85±0.05) and significant increase in coherence within theta and alpha frequency bands (FIG. 20E; 4-15.9 Hz; P<0.025, TGTC). Also, propofol peak max was characterized by decreased slow oscillation coherence (FIG. 20E; 0.1-1.7 Hz; P<0.025, TGTC). Our results are consistent with previous reports that coherent frontal beta-gamma oscillations and alpha oscillations are exhibited during propofol sedation and propofol-induced unconsciousness, respectively. Our results are also consistent with previous reports showing that incoherent slow oscillations are associated with propofol-induced unconsciousness.

Dexmedetomidine vs. Propofol Coherence

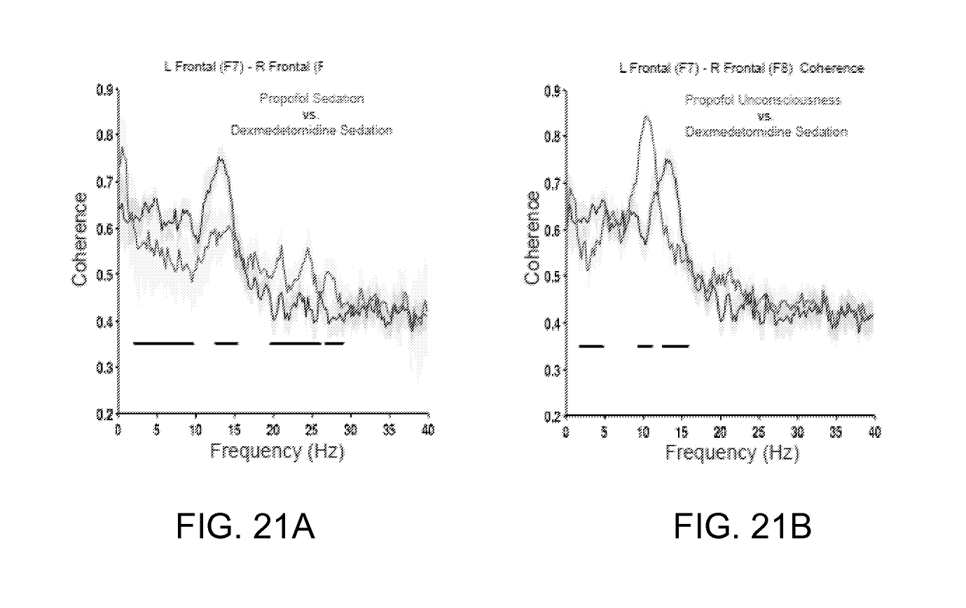

We next compared coherence patterns during dexmedetomidine sedation to those during propofol sedation and unconsciousness. Compared to propofol sedation, during dexmedeomidine sedation, the coherence was higher in the delta, theta, and alpha frequency bands, with a coherent dex-spindle peak (FIG. 21A; 2-10.5 Hz, 12.2-15.9 Hz; P<0.0005, TGTC). Also, consistent with the traveling peak, coherence was larger during propofol-induced sedation compared to dexmedetomidine sedation within beta frequency bands (FIG. 21A; 19.8-26.4 Hz, 26.9-29.3 Hz, P<0.0005, TGTC). Next, we compared the coherence patterns during dexmedetomidine sedation to propofol-induced unconsciousness. We found that dex-spindles and propofol-induced frontal alpha oscillations were distinctly different in terms of peak coherence and frequency (FIG. 21B). Coherence during propofol-induced unconsciousness was significantly higher at frequencies surrounding the alpha oscillation peak (FIG. 21B; 9.5-11.7 Hz; P<0.0005, TGTC). Coherence during dexmedetomidine-induced sedation was significantly higher within delta and theta bands as well as the frequency band surrounding the dex-spindles (FIG. 21B; 1.95-5.37 Hz, 12.7-16.6 Hz; P<0.0005, TGTC). Our results show again that the spindle-like EEG pattern induced by dexmedetomidine is a dynamic pattern distinct from the propofol-induced travelling peak and frontal alpha oscillations.

### Discussion

Although propofol- and dexmedetomidine-induced EEG signatures appear grossly similar, our analysis identifies distinct differences in the power spectrum and coherence that likely relate to the specific underlying mechanisms and clinical properties of these drugs. We briefly summarize our findings as follows:

(i) Similar to sleep spindles, dexmedetomidine sedation is characterized by spindles whose maximum power and coherence occur at ˜13-14 Hz. These dex-spindles are distinct in both the power spectrum and coherence from propofol traveling peak and alpha oscillations, which occur during propofol sedation and unconsciousness, respectively

(ii) Both dexmedetomidine sedation and propofol-induced unconsciousness are associated with slow oscillations characterized by increased power and reduced coherence at frequencies <1 Hz. However, slow oscillations during propofol-induced unconsciousness are an order of magnitude larger than that during dexmedetomidine sedation.

Slow oscillations have been proposed as a shared mechanism for unconsciousness during sleep and anesthesia. Since dexmedetomidine acts through neural circuits involved in the generation of NREM sleep, dexmedetomidine-induced slow waves are likely similar in nature to sleep slow waves. Both sleep slow waves and propofol-induced slow oscillations appear to have a local23 or spatially-asynchronous character that make them incoherent across different cortical regions. This is consistent with our finding that slow oscillation coherence decreases during both dexmedetomidine sedation and propofol-induced unconsciousness

At the neuronal level, slow oscillations are associated with an alternation between “ON” states where neurons are able to fire, and “OFF” states where neurons are silent. In sleep and under the alpha2 agonist xylazine, these “OFF” periods appear to be relatively brief, occupying a fraction of the slow oscillation period. In contrast, under propofol, these OFF periods are prolonged, occupying the majority of the slow oscillation period. This prolonged state of neuronal silence could explain why propofol produces a deeper state of unconsciousness from which patients cannot be aroused, compared to sleep or dexmedetomidine-induced sedation, where patients can be aroused to consciousness. Herein, we observed that propofol-induced slow oscillations were almost an order of magnitude larger than those during dexmedetomidine sedation. These much larger slow oscillations may explain why propofol OFF states appear prolonged compared to sleep or xylazine anesthesia. We speculate that the size of the propofol-induced slow oscillation, and the duration of the associated OFF states, could come from propofol's actions at GABAergic interneurons, which could help support larger slow waves and deeper levels of hyperpolarization required to sustain OFF states. Our results also suggest that the power or amplitude of slow oscillations could be used to distinguish between propofol-induced unconsciousness and sleep or sleep-like states such as dexmedetomidine-induced sedation.

The dex-spindle pattern that we have described herein has a frequency range and transient time-domain morphology that appears similar to sleep spindles. This suggests that the same thalamocortical circuit underlying sleep spindles could generate dex-spindles. Biophysical models have also established a thalamocortical basis for propofol-induced frontal alpha oscillations. This frontal alpha EEG activity is thought to contribute to alterations in consciousness by drastically restricting communication within frontal thalamocortical circuits from a wide to a narrow frequency band. They may also signify a change in anteriorposterior cortical coupling. Our results show that propofol-induced frontal alpha waves are larger and more coherent than dex-spindles, which may also explain why propofol is able to induce deeper levels of sedation and unconsciousness than dexmedetomidine. Our analysis suggest that these drugs are acting differently within the same underlying thalamocortical system. These differences may relate to the drugs underlying molecular and neuronal mechanisms. In particular, propofol's traveling peak dynamics, as well as its highly coherent frontal ˜10 Hz alpha oscillation, appear to be generated by enhanced GABA inhibition at cortical and thalamic interneurons. Meanwhile, dexmedetomidine appears to act through endogenous NREM sleep circuits, which may explain why dex-spindles appear similar in morphology to sleep spindles. Because of these differences between dex-spindles and propofol-induced frontal alpha, we suggest that the term “spindle” might be used specifically to refer to sleep and dexmedetomidine-induced spindles.

We have demonstrated distinct differences in the properties of slow oscillations and thalamocortical oscillations induced by dexmedetomidine and propofol. Given our knowledge of the molecular pharmacology, neural circuits, and clinical properties associated with these drugs, it is not surprising that these drugs have distinct EEG signatures. Moreover, based on our analysis and discussion, it is likely that these differences in EEG dynamics are directly related to underling differences in molecular and neural circuit mechanisms. While the EEG has historically been viewed within anesthesiology as a “black box,” our analysis suggests a powerful alternative: the EEG could be viewed within the existing mechanistic framework of pharmacology and clinical practice, enabling it to be monitored like other clinical physiological signals. The EEG signatures described here are relatively easy to compute and display in real-time, suggesting that it is possible to display these dynamics in a straightforward way as we do with other physiological signals.

### Sevoflurane-Induced EEG Patterns

Frontal electroencephalogram data were recorded using the Sedline brain function monitor (Masimo Corporation, Irvine Calif.). The EEG data were recorded with a pre-amplifier bandwidth of 0.5 to 92 Hz, sampling rate of 250 Hz, with 16-bit, 29 nV resolution. The standard Sedline Sedtrace electrode array records from electrodes located approximately at positions Fp1, Fp2, F7, and F8, with ground electrode at Fpz, and reference electrode approximately 1 cm above Fpz. Electrode impedance was less than 5 kΩ in each channel. An investigator experienced in reading the EEG (O.A.) visually inspected the data from each patient and selected EEG data free of noise and artifacts for analysis. EEG data segments were selected using information from the electronic anesthesia record. For each patient, 5-minute EEG segments representing the maintenance phase of general anesthesia were carefully selected. The data was selected from a time period after the initial induction bolus of an intravenous hypnotic and while the maintenance agent was stable.

Sevoflurane vs. Propofol Power Spectra

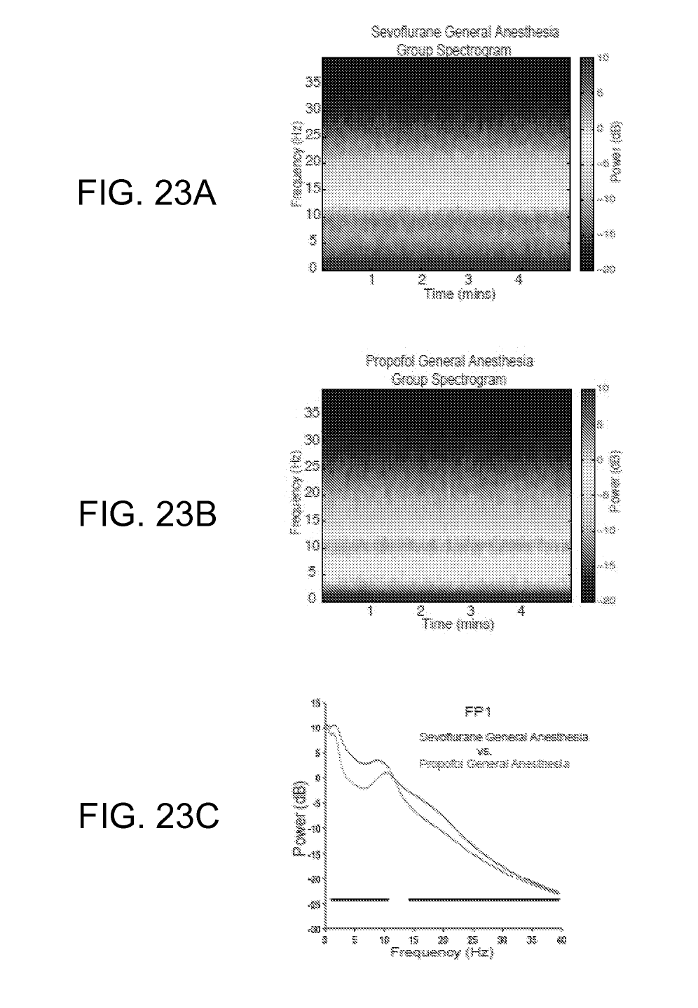

We observed similarities and differences in the spectrograms of the sevoflurane and propofol general anesthesia groups (FIGS. 23A, 23B). Both spectrograms were similarly characterized by large alpha band power. However, sevoflurane elicited higher power across the theta (4-8 Hz) and beta (12-25 Hz) frequency ranges (FIGS. 23A, 23B). Sevoflurane general anesthesia EEG power exhibited an alpha oscillation peak (mean±std; peak frequency, 9.2 Hz±0.84; peak power, 4.3 dB±3.5) that was only slightly different from the propofol general anesthesia alpha oscillation peak (peak frequency, 10.3 Hz±1.1; peak power, 2.1 dB±4.3). We next compared the EEG spectrum between these two groups and found significant differences in power across most frequencies between 0.4 and 40 Hz. Sevoflurane exhibited increased EEG power across a range of frequencies except at slow oscillations (<0.4 Hz) and the propofol alpha oscillation peak (FIG. 23C; 0.4-11.2 Hz, 14.7-40 Hz; P<0.001, TGTS). Our results show that, compared to propofol-induced unconsciousness, sevoflurane-induced unconsciousness was characterized by larger theta and beta oscillations, and similar slow and alpha oscillations.

Sevoflurane vs. Propofol Coherence

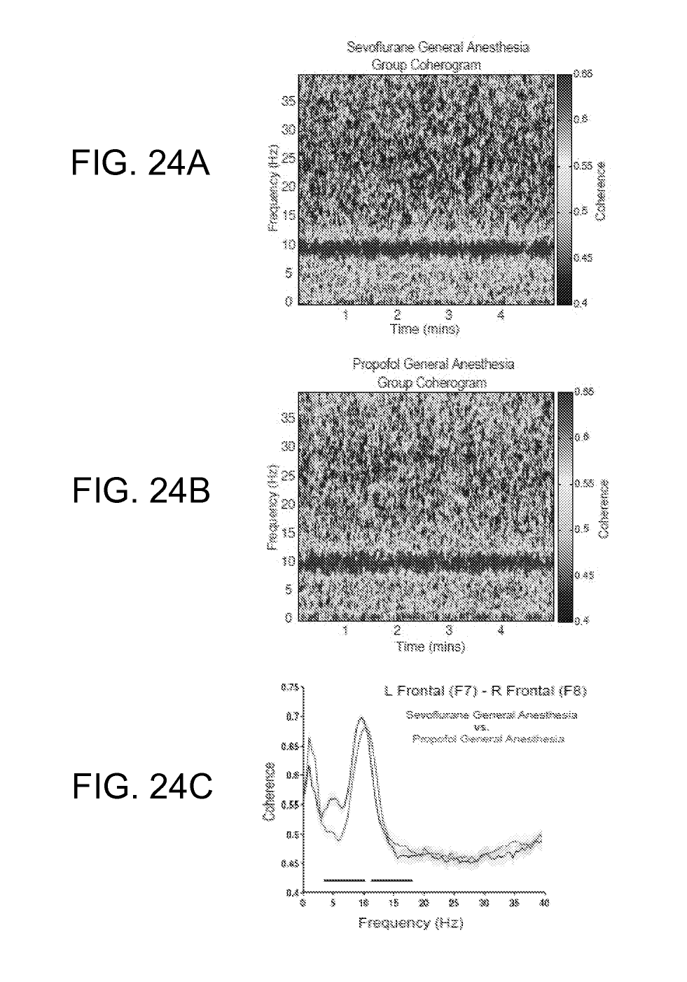

We also observed similarities and differences in coherograms of the sevoflurane and propofol general anesthesia groups (FIG. 24A, 24B). Both coherograms were similarly characterized by alpha band coherence, and the absence of slow oscillation coherence. However, the sevoflurane group coherogram also showed a coherence peak within the theta frequency range that was not evident in the propofol general anesthesia group (FIG. 24A, 24B; peak frequency, 4.9 Hz±0.6; peak coherence, 0.58±0.1). Sevoflurane GA EEG coherence exhibited an alpha oscillation peak (peak frequency, 9.8 Hz±0.91; peak coherence, 0.73±0.1) that was very similar to propofol GA alpha oscillation peak (peak frequency, 10.2 Hz±1.3; peak coherence, 0.71 dB±0.1). We next compared the EEG coherence between these two groups. We found that the sevoflurane and propofol coherence were qualitatively similar, showing a strong alpha peak, and lower slow oscillation peak. Sevoflurane exhibited increased EEG coherence across a range of theta and alpha frequencies (FIG. 24C; 3.41-10.7 Hz; TGTC, P<0.001) while propofol exhibited increased EEG coherence across a slightly different range of alpha and beta frequencies (FIG. 24C; 11.7-19.5 Hz; TGTC, P<0.001). Our results show that both sevoflurane and propofol GA are characterized by coherent frontal alpha oscillations with very similar peak frequencies and coherence. However, sevoflurane also showed coherent theta oscillations.

### Discussion

Although sevoflurane- and propofol-induced EEG signatures appear grossly similar, our analysis identifies a distinct difference in theta coherence that provides insight into the neural circuit mechanisms of sevoflurane. We briefly summarize our findings as follows:

(i) Similar to propofol-induced frontal alpha oscillations, sevoflurane is characterized by coherent alpha oscillations with similar maximum power and coherence occurring at ˜10-12 Hz;

(ii) Also similar to propofol, sevoflurane is associated with slow oscillations at frequencies <1 Hz; (iii) In contrast to propofol, sevoflurane is associated with increased power and coherence in the theta band.

The similarities between sevoflurane- and propofol-induced EEG dynamics are consistent with the notion that similar GABAergic neural circuit mechanisms are involved. This suggests that sevoflurane, like propofol, also induces highly structured thalamocortical oscillations that interfere with cortical information processing, as well as slow oscillations that fragment cortical activity. Preliminary studies suggest that these EEG signatures are also representative of the ether derivatives, isoflurane and desflurane, suggesting that these oscillatory patterns may be used as EEG signatures of general anesthesia induced loss of consciousness.

The coherent theta oscillations (˜5 Hz) characteristic of sevoflurane anesthesia, to our knowledge, have not been previously reported. Speculating on the possible significance of these theta oscillations, we note that pathological theta oscillations have been linked to dysfunction of low-threshold T-type calcium channels in thalamic neurons, leading to a thalamocortical dysrhythmia. Volatile anesthetics have been reported to modulate T-type calcium channels at clinically relevant concentrations in the dorsal root ganglia, hippocampal and thalamic relay neurons. These parallels lead us to hypothesize that sevoflurane-induced theta oscillations may be indicative of profound thalamic deafferentation. If true, this EEG signature along with those of slow and alpha oscillations may be useful to monitor depth of anesthesia in real-time. In the future, it would be important to study the spatiotemporal dynamics of this oscillatory dynamic with respect to depth of anesthesia.

Our findings suggest that propofol and sevoflurane, despite quantitative differences in the EEG power spectrum, both exhibit highly coherent frontal alpha oscillations that have been associated with entrainment of thalamocortical communications. However, sevoflurane also exhibits a theta-band coherence that was not present under propofol. Coherent theta oscillations are not generally present in the awake eyes closed state, implying that this coherence signature is sevoflurane induced. Also, we were able to observe these similarities and differences in EEG spectra and coherences in data recorded during routine care of patients undergoing a variety of surgical procedures, and under different co-administered medications, suggesting that these effects are robust.

The present analysis suggests a potential shared GABAergic mechanism for propofol and sevoflurane at clinically-relevant doses. Furthermore, it details EEG signatures that can be used to identify and monitor the shared and differential effects of anesthetic agents, providing a foundation for future analyses. The EEG recordings analyzed herein were obtained from frontal channels, and as a result, our analysis did not take into account anterior-posterior connectivity, which has been reported to contribute to cortical dynamics underlying anesthesia induced unconsciousness. Because this study was performed in the clinical setting, our inferences were restricted to a clinically unconscious state.

In summary, the practice of anesthesiology involves the direct pharmacological manipulation of the central nervous system to achieve the required combination of unconsciousness, amnesia, analgesia, and immobility with maintenance of physiological stability that define general anesthesia. Recent advances in neuroscience research methods are helping to refine the understanding of the neural circuit mechanisms of anesthesia-induced unconsciousness. Nonetheless, despite major advances in identifying common molecular and pharmacological principles that underlie anesthetic drugs, it is not yet apparent how actions at different molecular targets affect large-scale neural dynamics to produce unconsciousness. At the molecular level, general anesthetics modulate ion-channels in key regions of the brain and spinal cord to disrupt synaptic transmission, giving rise to distinct electroencephalogram (EEG) signatures. These ion-channels may include γ-Aminobutyric acid (GABA_{A}), glutamate, icotinic acetylcholine, glycine, potassium and serotonin. Given the diversity of receptor targets, a unitary hypothesis of the neural circuit mechanism underlying anesthesia-induced depression of consciousness does not seem likely.

Most studies have focused on a deep steady state of general anesthesia and have not used a systematic behavioral measure to track the transition into unconsciousness. This steady-state approach cannot distinguish between EEG patterns that are characteristic of a deeply anesthetized brain and those that arise at the onset of unconsciousness. For example, unconsciousness can occur in tens of seconds, but many neurophysiological features continue to fluctuate for minutes after induction and are highly variable between different levels of general anesthesia. As such, the relationships between stereotypical EEG patterns manifested by general anesthetics and altered arousal remain poorly understood. Therefore, identifying the specific dynamics associated with loss of consciousness (LOC) requires an examination of the transition into unconsciousness, linking neurophysiology with behavioral measures.

In one approach presented above, rapid propofol-induced unconsciousness was shown to cause the human brain to undergo an abrupt change in measured network dynamics using single units, local field potentials and intracranial probes. Neural dynamics were shown to be highly variable during the unconscious period, as spike rates and most oscillatory patterns continued to fluctuate for minutes after LOC. Spiking activity was constrained to brief time periods coupled to the phase of the slow oscillation, interrupting information processing within a cortical area. These brief activity periods were phase-shifted across cortex, limiting activity spatially, since different cortical areas are likely to be active at different times. By contrast, observed slow or low-frequency oscillations showed markedly distinct patterns, which developed simultaneously with LOC and maintained thereafter.

Also, slow oscillations were shown to fragment cortical processing by de-coupling cortical activity across space and time, disrupting the coordinated intra-cortical communication that is considered crucial for conscious processing. This asynchrony constrains neurons in different areas of the cerebral cortex, and possibly other brain structures, to fire in an asynchronous manner, disrupting or fragmenting coordinated brain activity, and examples shown herein indicate that slow oscillations prevent sustained localized information processing and communication between distant cortical areas, and thus may facilitate the breakdown of communication by isolating local cortical networks. As such, it was also demonstrated that slow or low-frequency synchrony may be used to distinguish between sedative states where patients can be aroused to consciousness, and general anesthetic states where patients cannot be aroused. In particular, slow or low-frequency synchrony was high in the sedative state, reduced in the unconscious general anesthetic state, and returned when patients emerged from general anesthesia.

In examples of propofol-induced anesthesia, visually evident decreases in slow, or low-frequency, oscillation coherence compared to propofol sedation were shown, suggesting that slow oscillation coherence decrease at deeper levels of unconsciousness. Showing qualitatively similar EEGs to propofol patterns, dexmedetomidine exhibited greater coherence in the range of slow oscillations and less coherence in the alpha/spindle frequency range, as compared to propofol. Moreover, data from sevoflurane also showed highly structured thalamocortical oscillations with spatiotemporal fragmentation, indicating that similar EEG signature dynamics are possible with other ether derivatives, such as isoflurane and desflurane.

Therefore, in accordance with the present disclosure, coherent and non-coherent slow or low-frequency oscillations, resulting from anesthesia-induced sedation and unconsciousness, may provide systems and methods with indicators, which are rigorously linked to basic neurophysiology of anesthesia-induced unconsciousness, for use in tracking or monitoring sedation or unconsciousness. Using measures of brain coherence and synchrony, and possibly other characteristics or indicators, systems and methods may be used to distinguish between sedative states of consciousness, where patients can be aroused by external stimuli, and general anesthetic states of consciousness, where patients cannot be aroused by external stimuli. In addition, measures of brain coherence and synchrony, and possibly other characteristics or indicators, may be used in systems and methods configured to predict, for example, when patients may emerge from general anesthesia, or predict when patients may enter a state of unconsciousness during induction of general anesthesia. Similarly, systems and methods in accordance with the present disclosure may also be used to determine when a patient's brain state and brain response to sedative drugs is changing during long-term sedation within an intensive care unit, or determine when a patient's brain state is changing due to metabolic or infectious disease states during intensive care.

As described, anesthesia-induced unconsciousness may be associated with two specific states of brain dynamics. The first is a highly synchronous oscillation in the alpha or spindle band involving the thalamus and frontal cortex. The second consists of asynchronous <1 Hz slow oscillations. These oscillations generate large electromagnetic fields that can be recorded at the scalp in the form of electroencephalogram. The coherence and cohereogram methods described here provide a means of identifying these thalamocortical and asynchronous slow oscillations. In particular, coherence or cohereogram can be used to improve monitoring and quantification of these anesthesia-induced brain dynamics.

Specifically, it can be difficult at times to clearly identify the frontal alpha or spindle oscillations, which are anesthesia-induced thalamocortical oscillations associated with the unconscious state, just from looking at the spectrogram. The visibility of these oscillations can depend on how the spectrogram is scaled, and the structure of oscillations in the beta, alpha, theta, delta, and slow bands can be difficult to discern (FIG. 22A). For instance, with inhaled anesthetics such as Sevoflurane (FIGS. 22A, 23A), Isoflurane, or Desflurane, for example, the spectrum across the beta, alpha, theta, delta, and slow bands can fill-in and appear as a continuous band. In comparison, coherence information or cohereograms clearly show the presence of the 10 Hz alpha oscillation under sevoflurane (FIGS. 24A, 24B, and 24C). Similarly, with dexmedetomidine, spindle oscillations can be difficult to discern with the spectrum or spectrogram alone (FIG. 15B). However, the spindle oscillations become much clearer when examined using coherence information or cohereograms (FIGS. 19B and 19C). Thus, the coherence and cohereogram information, in accordance with the present disclosure, provide a clearer view of the frontal alpha and spindle oscillations that reflect thalamocortical oscillations associated with the unconscious state.

In addition, it can also be difficult at times to clearly identify the slow oscillations induced by anesthetic drugs just from looking at the spectrum or spectrogram. This is because low-frequency power, less than approximately 1 Hz, may generally be present in the baseline conscious state (FIGS. 15A and 16A). Anesthesia-induced slow oscillations associated with unconsciousness, as described, are asynchronous across different areas of the cerebral cortex. Furthermore, coherence provides a means to quantify the extent to which oscillations are synchronized (FIGS. 18B and 18C). Also, the asynchronous slow oscillations can be clearly discerned from the coherence information and cohereograms. For dexmedetomidine, in FIGS. 15A, 15B, and 15C there is visible power in the slow oscillation band during both baseline and sedated states. Although the slow oscillation power is statistically significantly different between the two states (FIG. 15C), the difference is difficult to discern. However, when examined using coherence or cohereogram information, the dexmedetomidine-induced asynchronous slow oscillation is clearly visible, in the form of a reduced coherence. This is clearly visible in comparing FIGS. 19A and 19B, which show how coherence in the <1 Hz band decreases under the sedative state. It is also clearly visible in FIG. 19C, in comparing the <1 Hz coherence during baseline and sedative states. This scenario is also clearly evident in the case of propofol. In FIG. 16A, 16B, and 16C, slow oscillation power <1 Hz is visible across baseline, sedated, and unconscious states. When viewed in terms of coherence information or cohereograms, as in FIGS. 20C and 20E, the loss of <1 Hz coherence is clearly visible in the unconscious state. Thus, the coherence and cohereogram provide a means to more clearly identify anesthesia-induced asynchronous slow oscillations associated with the unconscious state.

Thus, a clinician could concurrently view the spectrogram and cohereogram, or the cohereogram alone, and seek to maintain a strong coherence in the alpha or spindle band. Changes in the alpha or spindle band coherence could indicate changing drug levels, or the changes in the patient's state of arousal or consciousness. In such cases, the clinician could adjust the drug dose to maintain the alpha or spindle band coherence. Similarly, the clinician could seek to maintain a low coherence in the slow oscillation band. Changes in the slow oscillation coherence could indicate changing drug levels, or the changes in the patient's state of arousal or consciousness. In such cases, the clinician could adjust the drug dose to maintain reduced slow oscillation coherence. If the clinician were interested in having the patient recover consciousness, or have the patient go to a state where they could be more easily aroused or more easily recover consciousness, or one where the patient is conscious but sedated, the coherence could be used to help achieve these states as well. For instance, the absence of alpha or spindle band coherence, or the presence of slow oscillation coherence, could be used to determine whether the patient were in a sedated state

This novel approach may shift the focus of anesthesiology towards understanding the neurophysiology and neuroanatomical basis of brain states created by anesthetic drugs, and may position anesthesiologists to make new and important contributions to clinical practice and neuroscience research by directly furthering knowledge of the neural bases of sleep, arousal and pathological states. Although at present the mechanisms underlying slow oscillations are unclear, slow oscillations, however, may play a key role in the modulation of higher frequency oscillations, and hence spatiotemporal fragmentation of slow oscillations in all these states may help explain the impairment of cortical integration.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

Embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the disclosures described herein can be embodied within a form that does not provide all of the features and benefits set forth herein, as some features can be used or practiced separately from others. The scope of certain disclosures disclosed herein is indicated by the appended claims rather than by the foregoing description. All changes which come within the meaning and range of equivalency of the claims are to be embraced within their scope.

1. A system for monitoring a patient experiencing an administration of at least one drug having anesthetic properties, the system comprising:
at least one sensor configured to acquire physiological data from the patient;
a user interface configured to receive an indication of at least one of a characteristic of the patient and the at least one drug having anesthetic properties;
at least one processor configured to:
receive the physiological data from the plurality of sensors and the indication from the user interface;
separate, from the physiological data, a plurality of low frequency signals;
determine, from the plurality of low frequency signals, at least one of coherence information and synchrony information;
identify, using the at least one of the coherence information and the synchrony information, spatiotemporal signatures indicative of at least one of a current state and a predicted future state of the patient consistent with administration of at least one drug having anesthetic properties; and
generate a report indicating at least one of the current state and the predicted future state of the patient induced by the drug.

2. The system of claim 1 wherein the plurality of low frequency signals are within a frequency range between 0.1 Hz and 1 Hz.

3. The system of claim 1 wherein the at least one processor is further configured to use the current state, determined spatiotemporal signatures, and indication are in a model to determine the predicted future state of the patient.

4. The system of claim 1 wherein the processor is configured to assemble the physiological data into sets of time-series data and transform each set of time-series data into a spectrogram to determine at least one of the current state and the predicted future state of the patient.

5. The system of claim 1 wherein the processor is configured to assemble the physiological data into sets of time-series data and each set of time-series data is transformed into a coherogram to determine at least one of the current state and the predicted future state of the patient.

6. The system of claim 1 wherein the processor is configured to perform a phase analysis on the plurality of low frequency signals to measure a time-resolved phase-coupling and identify synchrony information corresponding to at least one of the current state and the predicted future state of the patient.

7. The system of claim 1 wherein the processor is configured to perform a coherence analysis on the plurality of low frequency signals to measure a frequency-dependent covariance and identify coherence information corresponding to at least one of the current state and the predicted futures state of the patient.

8. The system of claim 1 wherein the indication of at least one of a characteristic of the patient includes at least one of an age of the patient, drug administration information including at least one of drug timing, drug dose, and drug administration rate, and the at least one drug having anesthetic properties is selected from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, and Alfentanil.

9. The system of claim 1 wherein the processor is configured to perform a dynamic processing method to characterize the patient as exhibiting a predetermined behavioral dynamic, wherein the behavioral dynamic includes at least one of a loss of consciousness and recovery of consciousness.

10. The system of claim 1 wherein the report indicates spatiotemporal activity at different states of the patient receiving the drug.

11. A method for monitoring a patient experiencing an administration of at least one drug having anesthetic properties, the method comprising:
arranging at least one sensor configured to acquire physiological data from a patient;
reviewing the physiological data from the at least one sensor;
identifying, from the physiological data, a plurality of low frequency signals;
determining, from the plurality of low frequency signals, at least one of coherence information and synchrony information;
identifying, using the at least one of the coherence information and the synchrony information, spatiotemporal signatures indicative of at least one of a current state and a predicted future state of the patient consistent with the administration of at least one drug having anesthetic properties; and
generating a report indicating at least one of the current state and the predicted future state of the patient induced by the drug.

12. The method of claim 11 wherein the low frequency signals are within a frequency range between 0.1 Hz and 1 Hz.

13. The method of claim 11 further comprising using the current state, determined spatiotemporal signatures, and the indication in a model to determine the predicted future state of the patient.

14. The method of claim 11 further comprising transforming the physiological data into a spectrogram and analyzing the spectrogram to determine at least one of the current state and the predicted future state of the patient.

15. The method of claim 11 further comprising transforming the physiological data into a coherogram and analyzing the coherogram to determine at least one of the current state and the predicted future state of the patient.

16. The method of claim 11 further comprising performing a phase analysis on the plurality of low frequency signals to measure a phase-coupling to identify synchrony information corresponding to at least one of the current state and the predicted future state of the patient.

17. The method of claim 11 further comprising performing a coherence analysis on the plurality of low frequency signals to measure a frequency-dependent covariance to identify coherence information corresponding to at least one of the current state and the predicted futures state of the patient.

18. The method of claim 11 wherein the at least one drug having anesthetic properties is selected from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, and Alfentanil.

19. The method of claim 11 further comprising implementing a dynamic processing method to characterize the patient as exhibiting a predetermined behavioral dynamic, and wherein the behavioral dynamic includes at least one of a loss consciousness and recovery of consciousness.

20. The method of claim 11 wherein the report indicates spatiotemporal activity at different states of the patient receiving the drug.

21. The method of claim 11 wherein identifying spatiotemporal signatures includes generating at least one of a spectrogram and a coherogram using multitaper method.
